SEQUENCE LISTING (14 sequences)

SEQ ID NO: 1 - protein, 1368 aa, Streptococcus pyogenes [sequence omitted]

SEQ ID NO: 2 - protein, 16 aa, Streptococcus pyogenes
RENQTTQKGQKNSRER

SEQ ID NO: 3 - protein, 16 aa, Streptococcus pyogenes
VDHIVPQSFLKDDSID

SEQ ID NO: 4 - protein, 15 aa, Streptococcus pyogenes
LDKAGFIKRQLVETR

SEQ ID NO: 5 - protein, 16 aa, Artificial Sequence
  Synthetic sequence
REEQTTRQGQDNSREK

SEQ ID NO: 6 - protein, 16 aa, Artificial Sequence
  Synthetic sequence
RDEQTTGEGQKNSREK

SEQ ID NO: 7 - protein, 16 aa, Artificial Sequence
  Synthetic sequence
VDHIVPRSFMTDNSFD

SEQ ID NO: 8 - protein, 16 aa, Artificial Sequence
  Synthetic sequence
VDHILPRSYMKDDSFD

SEQ ID NO: 9 - protein, 16 aa, Artificial Sequence
  Synthetic sequence
VDHIIPRSFLRNDSLD

SEQ ID NO: 10 - protein, 16 aa, Artificial Sequence
  Synthetic sequence
VDHVIPQSFMTDDSIE

SEQ ID NO: 11 - protein, 15 aa, Artificial Sequence
  Synthetic sequence
LEKQGFVKRQLMETR

SEQ ID NO: 12 - protein, 15 aa, Artificial Sequence
  Synthetic sequence
LDEQRWIKRQLVETQ

SEQ ID NO: 13 - protein, 15 aa, Artificial Sequence
  Synthetic sequence
LDEARWVKRQLMETR

SEQ ID NO: 14 - protein, 161 aa, Streptococcus pyogenes
RENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETR

(19) United States
(12) Patent Application Publication
(10) Pub. No.: US 2024/0043820 A1
(43) Pub. Date: Feb. 8, 2024

(54) ENZYME VARIANTS

(71) Applicants: THE UNIVERSITY OF WESTERN AUSTRALIA, Crawley (AU); YEDA RESEARCH AND DEVELOPMENT CO. LTD., Rehovot (IL)

(72) Inventors: Oliver Rackham, Stirling (AU); Aleksandra Filipovska, Stirling (AU); Pascal Vos, South Perth (AU); Sarel-Jacob Fleishman, Rehovot (IL); Olga Khersonsky, Rehovot (IL)

(21) Appl. No.: 18/266,385

(22) PCT Filed: Dec. 13, 2021
(86) PCT No.: PCT/AU2021/051484

§ 371 Date: Jun. 9, 2023

(30) Foreign Application Priority Data
Dec. 11, 2020 (AU) 2020904609

(51) Int. Cl. C12N 9/22; C12N 15/63

(52) U.S. Cl. CPC C12N 9/22; C12N 15/63; C12N 2310/20

(57) ABSTRACT

Provided herein are Cas9 proteins comprising SEQ ID NO:1 or a sequence at least 80% identical thereto, wherein: the amino acid residues at positions 765 to 780 are replaced by the amino acid sequence of SEQ ID NO:5 or SEQ ID NO:6; the amino acid residues at positions 838 to 853 are replaced by the amino acid sequence of SEQ ID NO:7, SEQ ID NO:8, SEQ ID NO:9 or SEQ ID NO:10; and/or the amino acid residues at positions 911 to 925 are replaced by the amino acid sequence of SEQ ID NO:11, SEQ ID NO:12 or SEQ ID NO:13. Also provided are Cas9 proteins comprising an HNH domain comprising the amino acid sequence of SEQ ID NO:14 or a sequence at least 80% identical thereto, wherein: the amino acid residues at positions 1 to 16 are replaced by the amino acid sequence of SEQ ID NO:5 or SEQ ID NO:6; the amino acid residues at positions 74 to 89 are replaced by the amino acid sequence of SEQ ID NO:7, SEQ ID NO:8, SEQ ID NO:9 or SEQ ID NO:10; and/or the amino acid residues at positions 147 to 161 are replaced by the amino acid sequence of SEQ ID NO:11, SEQ ID NO:12 or SEQ ID NO:13.

### FIELD OF THE ART

The present disclosure relates generally to Cas9 proteins with improved on-target activity, useful for clinical and research applications.

### BACKGROUND

Precision genome engineering via the clustered regularly interspaced short palindromic repeats/CRISPR-associated protein (CRISPR/Cas) system has revolutionized molecular biology. This specific and adaptable method for genome engineering typically utilizes a two-component system consisting of a Cas endonuclease and guide RNA (gRNA), which can be designed to target essentially any genomic locus and generate double-strand breaks. The gRNA comprises a mature CRISPR RNA (crRNA) and a trans-activating crRNA (tracrRNA) that are often combined into a single guide RNA (sgRNA) molecule. The Cas-gRNA complex binds a DNA sequence complementary to a sequence in the crRNA, lying adjacent to a Cas-ortholog specific PAM (protospacer adjacent motif) sequence which is required for enzymatic cleavage of its target. Cas9-generated double strand breaks are subsequently repaired via non-homologous end-joining or homology-directed repair, thereby editing the genome.

The most widely used Cas endonuclease in CRISPR/Cas genomic engineering applications is Cas9 from Streptococcus pyogenes (SpCas9), used, for example, in target gene disruption, transcriptional repression and activation, epigenetic modulation, and single nucleotide conversion in a wide variety of cell types and organisms. SpCas9 recognizes the relatively abundant PAM sequence NGG. Cas9 contains two catalytic (nuclease) domains, the modular RuvC-like domain and the HNH-like domain. Each domain cleaves one of the target DNA strands, resulting in a blunt-ended double strand break or short overhang upstream of the PAM motif.

Existing CRISPR/Cas9 systems suffer from several problems, including low activity of Cas9 and a high frequency of off-target cleavage. In many therapeutic scenarios the level of Cas9 activity, or the rate at which mutagenesis occurs, is the principal limiting factor. Previously reported Cas9 mutations designed to lower Cas9 off-target cleavage have often resulted in a decreased affinity for its target sequence and a reduced mutagenesis rate. Accordingly, there is a need in the art to develop new Cas9 variants with higher activity and higher catalytic efficiency.

### SUMMARY OF THE DISCLOSURE

The present disclosure is predicated on the inventors' engineering, using computational mutagenesis of the HNH domain of SpCas9 coupled with a rapid, quantitative yeast screening system, to generate SpCas9 variants with improved activity and higher mutagenesis rates.

Accordingly, in one aspect, the present disclosure provides an isolated Cas9 protein comprising SEQ ID NO:1 or a sequence at least about 80% identical thereto, wherein the amino acid residues at positions 765 to 780 are replaced by the amino acid sequence of SEQ ID NO:5 or SEQ ID NO:6.

Another aspect of the present disclosure provides an isolated Cas9 protein comprising SEQ ID NO:1 or a sequence at least about 80% identical thereto, wherein the amino acid residues at positions 838 to 853 are replaced by the amino acid sequence of SEQ ID NO:7, SEQ ID NO:8, SEQ ID NO:9 or SEQ ID NO:10.

In a particular exemplary embodiment, the Cas9 protein comprises SEQ ID NO:1 or a sequence at least about 80% identical thereto, wherein the amino acid residues at positions 838 to 853 are replaced by the amino acid sequence of SEQ ID NO:8.

Another aspect of the present disclosure provides an isolated Cas9 protein comprising SEQ ID NO:1 or a sequence at least about 80% identical thereto, wherein the amino acid residues at positions 911 to 925 are replaced by the amino acid sequence of SEQ ID NO:11, SEQ ID NO:12 or SEQ ID NO:13.

Another aspect of the present disclosure provides an isolated Cas9 protein comprising SEQ ID NO:1 or a sequence at least about 80% identical thereto, wherein the amino acid residues at positions 765 to 780 are replaced by the amino acid sequence of SEQ ID NO:5 or SEQ ID NO:6 and the amino acid residues at positions 838 to 853 are replaced by the amino acid sequence of SEQ ID NO:7, SEQ ID NO:8, SEQ ID NO:9 or SEQ ID NO:10.

Another aspect of the present disclosure provides an isolated Cas9 protein comprising SEQ ID NO:1 or a sequence at least about 80% identical thereto, wherein the amino acid residues at positions 765 to 780 are replaced by the amino acid sequence of SEQ ID NO:5 and the amino acid residues at positions 911 to 925 are replaced by the amino acid sequence of SEQ ID NO:11 or SEQ ID NO:13.

Another aspect of the present disclosure provides an isolated Cas9 protein comprising SEQ ID NO:1 or a sequence at least about 80% identical thereto, wherein the amino acid residues at positions 765 to 780 are replaced by the amino acid sequence of SEQ ID NO:6 and the amino acid residues at positions 911 to 925 are replaced by the amino acid sequence of SEQ ID NO:11 or SEQ ID NO:12.

Another aspect of the present disclosure provides an isolated Cas9 protein comprising SEQ ID NO:1 or a sequence at least about 80% identical thereto, wherein the amino acid residues at positions 838 to 853 are replaced by the amino acid sequence of SEQ ID NO:7, SEQ ID NO:8, SEQ ID NO:9 or SEQ ID NO:10 and the amino acid residues at positions 911 to 925 are replaced by the amino acid sequence of SEQ ID NO:11, SEQ ID NO:12 or SEQ ID NO:13.

Another aspect of the present disclosure provides an isolated Cas9 protein comprising SEQ ID NO:1 or a sequence at least about 80% identical thereto, wherein: the amino acid residues at positions 765 to 780 are replaced by the amino acid sequence of SEQ ID NO:5 or SEQ ID NO:6; the amino acid residues at positions 838 to 853 are replaced by the amino acid sequence of SEQ ID NO:7, SEQ ID NO:8, SEQ ID NO:9 or SEQ ID NO:10; and the amino acid residues at positions 911 to 925 are replaced by the amino acid sequence of SEQ ID NO:11, SEQ ID NO:12 or SEQ ID NO:13.

In an exemplary embodiment, the amino acid residues at positions 765 to 780 are replaced by the amino acid sequence of SEQ ID NO:5, the amino acid residues at positions 838 to 853 are replaced by the amino acid sequence of SEQ ID NO:7, and the amino acid residues at positions 911 to 925 are replaced by the amino acid sequence of SEQ ID NO:13. In another exemplary embodiment, the amino acid residues at positions 765 to 780 are replaced by the amino acid sequence of SEQ ID NO:6, the amino acid residues at positions 838 to 853 are replaced by the amino acid sequence of SEQ ID NO:7, and the amino acid residues at positions 911 to 925 are replaced by the amino acid sequence of SEQ ID NO:11. In another exemplary embodiment, the amino acid residues at positions 765 to 780 are replaced by the amino acid sequence of SEQ ID NO:6, the amino acid residues at positions 838 to 853 are replaced by the amino acid sequence of SEQ ID NO:8, and the amino acid residues at positions 911 to 925 are replaced by the amino acid sequence of SEQ ID NO:11. In another exemplary embodiment, the amino acid residues at positions 765 to 780 are replaced by the amino acid sequence of SEQ ID NO:6, the amino acid residues at positions 838 to 853 are replaced by the amino acid sequence of SEQ ID NO:8, and the amino acid residues at positions 911 to 925 are replaced by the amino acid sequence of SEQ ID NO:12.

In accordance with the above aspects, the Cas9 protein may be derived from the Cas9 protein of Streptococcus pyogenes.

Another aspect of the present disclosure provides an isolated Cas9 protein comprising an HNH domain comprising the amino acid sequence of SEQ ID NO:14 or a sequence at least about 80% identical thereto, wherein: the amino acid residues at positions 1 to 16 are replaced by the amino acid sequence of SEQ ID NO:5 or SEQ ID NO:6; the amino acid residues at positions 74 to 89 are replaced by the amino acid sequence of SEQ ID NO:7, SEQ ID NO:8, SEQ ID NO:9 or SEQ ID NO:10; and/or the amino acid residues at positions 147 to 161 are replaced by the amino acid sequence of SEQ ID NO:11, SEQ ID NO:12 or SEQ ID NO:13.

In a particular exemplary embodiment, the Cas9 protein comprises an HNH domain comprising the amino acid sequence of SEQ ID NO:14 or a sequence at least about 80% identical thereto, wherein the amino acid residues at positions 74 to 89 are replaced by the amino acid sequence of SEQ ID NO:8.

In accordance with the above aspect, the HNH domain may be derived from the Cas9 protein of Streptococcus pyogenes.

In another aspect, the present disclosure provides an isolated polynucleotide encoding a Cas9 protein as described herein.

In another aspect, the present disclosure provides a vector comprising the polynucleotide as described herein.

In another aspect, the present disclosure provides a complex comprising a Cas9 protein as described herein and a guide RNA (gRNA) bound to the HNH domain of the Cas9 protein.

### BRIEF DESCRIPTION OF THE DRAWINGS

Embodiments of the disclosure are described herein, by way of non-limiting example only, with reference to the accompanying drawings.

FIG. 1. Cas9 efficacy screen in Saccharomyces cerevisiae. (A) Schematic representation of the vectors used in the screening system described herein. (B) Dotting of Cas9 vectors and the control (Empty) with the gRNAs ADE2, HIS3 and CAN1. (C) Schematic representation of the Cas9 inhibitor system described herein. (D) Dotting of SpCas9 with Cas9 inhibitor system. (E) Survival assay of SpCas9 compared to a negative control.

FIG. 2. Design and quantification of Funclib mutants. (A-D) 3D representation of the three targeted regions in the HNH domain. (A) Overview of the residues that interact with the DNA or RNA. (B) Region 1 depicted in red. (C) Region 2 depicted in the colour marine. (D) Region 3 depicted in the colour violet. (E) List of the mutations for each of the regions. (F) Functional screen of the Funclib mutants in the absence of inhibitors. (G-L) Quantitative survival assays in the presence of inhibitors for the active mutants of (G-H) region 1, (I-J) region 2 and (K-L) region 3. CFU (colony forming units). In (G), for both ADE2 and HIS3, from left to right: WT, Mut 1.4, Mut 1.5, Mut 1.8. In (H), for both ADE2 and HIS3, from left to right: WT, Mut 1.4, Mut 1.5. In (I), for both ADE2 and HIS3, from left to right: WT, Mut 2.1, Mut 2.2, Mut 2.4, Mut 2.6, Mut 2.7, Mut 2.8, Mut 2.10. In (J), for ADE2, from left to right: WT, Mut 2.4. In (J), for HIS3, from left to right: WT, Mut 2.1, Mut 2.2, Mut 2.4, Mut 2.10. In (K), for both ADE2 and HIS3, from left to right: WT, Mut 3.2, Mut 3.3, Mut 3.4, Mut 3.7, Mut 3.8, Mut 3.9, Mut 3.10. In (L), from left to right: WT, Mut 3.8, Mut 3.9, Mut 3.10.

FIG. 3. Enhancing the efficacy of Cas9 by combining multiple Funclib mutants. (A-D) Survival assays of the combined mutants using the qualitative assay described herein. (A) Combined mutants of mut 1.4. (B) Combined mutants of mut 1.5. (C) Combined mutants of mut 2.1 and mut 2.2. (D) Combined mutants of mut 2.4 and mut 2.10. (E-H) Comparison of double mutant activity relative to their individual counterparts. (E) Comparison of combinations mutants based of mut 1.4. (F) Comparison of combinations mutants based of mut 1.5. (G) Comparison of combination mutants based of mut 2.1 and mut 2.2. (H) Comparison of combination mutants based of mut 2.4 and mut 2.10. (I-L) Quantitative survival assays of working mutant combinations. (I) Quantification of combinations of mut 1.4. (J) Quantification of combinations of mut 1.5. (K) Quantification of combinations of mut 2.1 and mut 2.2. (L) Quantification of combinations of mut 2.4 and mut 2.10. In (I), for both ADE2 and HIS3, from left to right: WT, Mut 1.4-2.1, Mut 1.4-2.2, Mut 1.4-2.4, Mut 1.4-2.10, Mut 1.4-3.8, Mut 1.4-3.9, Mut 1.4-3.10. In (J), for both ADE2 and HIS3, from left to right: WT, Mut 1.5-2.1, Mut 1.5-2.2, Mut 1.5-2.4, Mut 1.5-2.10, Mut 1.5-3.8, Mut 1.5-3.9, Mut 1.5-3.10. In (K), for both ADE2 and HIS3, from left to right: WT, Mut 2.1-3.8, Mut 2.1-3.9, Mut 2.1-3.10, Mut 2.2-3.8, Mut 2.2-3.9, Mut 2.2-3.10. In (L), for both ADE2 and HIS3, from left to right: WT, Mut 2.4-3.8, Mut 2.4-3.9, Mut 2.4-3.10, Mut 2.10-3.9, Mut 2.10-3.10.

FIG. 4. Hyperactive Cas9 enzymes effectively generate large and complex mutations in mammalian cells. (A) Percentage of indels introduced into the VEGFA gene by engineered Cas9 enzymes in HEK293T cells. (B) Fold change in Cas9 activity of selected mutants relative to wild-type Cas9. (C) Engineered Cas9 enzymes produce more complex, multiply edited mutations. (D) Engineered Cas9 enzymes introduce significantly larger deletions. Error bars: s.e.m. for n=3. FDR-adjusted p-value: *p<0.05, **p<0.01, ***p<0.001. In (A), (B) and (D), from left to right: WT, Mut 1.4, Mut 2.2, Mut 2.4, Mut 3.9, Mut 1.4-2.1, Mut 1.5-2.2, Mut 1.5-2.4, Mut 2.1-3.9, Mut 2.2-3.9, and Mut 2.4-3.9.

FIG. 5. Complexity of mutations introduced by engineered Cas9 enzymes in human cells. (A) Distribution of the different CC levels in VEGFA alleles upon editing by engineered Cas9 enzymes. (B) Occurrence of mutations that cause a frameshift, classified by particular mutation type. Error bars: s.e.m. of n=3. FDR-adjusted p-value: *p<0.05, **p<0.01, ***530 p<0.001. In (B), from left to right: WT, Mut 1.4, Mut 2.2, Mut 2.4, Mut 3.9, Mut 1.4-2.1, Mut 1.5-2.2, Mut 1.5-2.4, Mut 2.1-3.9, Mut 2.2-3.9, and Mut 2.4-3.9.

FIG. 6. (A) Enhanced activity does not consistently increase off-target DNA editing (at five known off-target sites for VEGF gRNA, named OFF22, OFF14, OFF10, OFFS-1 and OFFS-2), determined by the percentage of indels and (B) the fold change relative to wild-type Cas9. (C) The occurrence of different editing events varies between Cas9 variants and off-target sites. Error bars: s.e.m. for n=3. FDR-adjusted p-value: *p<0.05, **p<0.01, ***. In (A), (B) and (C), for each off-target site (OFF), from left to right: WT, Mut 2.2, and Mut 2.2-3.9.

FIG. 7 Enhanced base editing at HEK site 2 by incorporating the Mut 2.2 (TurboCas9) sequences into an adenine base editor (ABE) system. (A) The HEK site 2 target region gRNA and the possible A to G edits are shown schematically and detected edits are graphed for each nucleotide position. (B) Base editing at the FANCF site 1 target site.

Amino acid sequences described herein are referred to by a sequence identifier number (SEQ ID NO). Sequences are provided in Table 1 below and appear in the Sequence Listing appearing at the end of the specification.

TABLE 1
Amino acid sequences described herein

| SEQ ID | | |
|---|---|---|
| NO: | SEQUENCE | Description |
| 1 | MDKKYSIGLDIGTNSVGWAVITDEYKVPSKKFKVLGNTDRHSIKKN | Wild-type |
| | LIGALFDSGETAEATRLKRTARRRYTRRKNRICYLQEIFSNEMAKVD | SpCas9 |
| | DSFFHRLEESFLVEEDKKHERHPIFGNIVDEVAYHEKYPTIYHLRKKL | (UniProt Q99ZW2) |
| | VDSTDKADLRLIYLALAHMIKFRGHFLIEGDLNPDNSDVDKLFIQLV | |
| | QTYNQLFEENPINASGVDAKAILSARLSKSRRLENLIAQLPGEKKNG | |
| | LFGNLIALSLGLTPNFKSNFDLAEDAKLQLSKDTYDDDLDNLLAQIG | |
| | DQYADLFLAAKNLSDAILLSDILRVNTEITKAPLSASMIKRYDEHHQ | |
| | DLTLLKALVRQQLPEKYKEIFFDQSKNGYAGYIDGGASQEEFYKFIK | |
| | PILEKMDGTEELLVKLNREDLLRKQRTFDNGSIPHQIHLGELHAILRR | |
| | QEDFYPFLKDNREKIEKILTFRIPYYVGPLARGNSRFAWMTRKSEETI | |
| | TPWNFEEVVDKGASAQSFIERMTNFDKNLPNEKVLPKHSLLYEYFT | |
| | VYNELTKVKYVTEGMRKPAFLSGEQKKAIVDLLFKTNRKVTVKQL | |
| | KEDYFKKIECFDSVEISGVEDRFNASLGTYHDLLKIIKDKDFLDNEEN | |
| | EDILEDIVLTLTLFEDREMIEERLKTYAHLFDDKVMKQLKRRRYTG | |
| | WGRLSRKLINGIRDKQSGKTILDFLKSDGFANRNFMQLIHDDSLTFK | |
| | EDIQKAQVSGQGDSLHEHIANLAGSPAIKKGILQTVKVVDELVKVM | |
| | GRHKPENIVIEMARENQTTQKGQKNSRERMKRIEEGIKELGSQILKE | |
| | HPVENTQLQNEKLYLYYLQNGRDMYVDQELDINRLSDYDVDHIVP | |
| | QSFLKDDSIDNKVLTRSDKNRGKSDNVPSEEVVKKMKNYWRQLLN | |
| | AKLITQRKFDNLTKAERGGLSELDKAGFIKRQLVETRQITKHVAQIL | |
| | DSRMNTKYDENDKLIREVKVITLKSKLVSDFRKDFQFYKVREINNY | |
| | HHAHDAYLNAVVGTALIKKYPKLESEFVYGDYKVYDVRKMIAKSE | |
| | QEIGKATAKYFFYSNIMNFFKTEITLANGEIRKRPLIETNGETGEIVW | |
| | DKGRDFATVRKVLSMPQVNIVKKTEVQTGGFSKESILPKRNSDKLIA | |
| | RKKDWDPKKYGGFDSPTVAYSVLVVAKVEKGKSKKLKSVKELLGI | |
| | TIMERSSFEKNPIDFLEAKGYKEVKKDLIIKLPKYSLFELENGRKRML | |
| | ASAGELQKGNELALPSKYVNFLYLASHYEKLKGSPEDNEQKQLFVE | |
| | QHKHYLDEIIEQISEFSKRVILADANLDKVLSAYNKHRDKPIREQAEN | |
| | IIHLFTLTNLGAPAAFKYFDTTIDRKRYTSTKEVLDATLIHQSITGLYE | |
| | TRIDLSQLGGD | |
| 2 | RENQTTQKGQKNSRER | SpCas9 HNH |
| | | domain region 1 |
| 3 | VDHIVPQSFLKDDSID | SpCas9 HNH |
| | | domain region 2 |
| 4 | LDKAGFIKRQLVETR | SpCas9 HNH |
| | | domain region 3 |
| 5 | REEQTTRQGQDNSREK | Mut 1.4 |
| 6 | RDEQTTGEGQKNSREK | Mut 1.5 |
| 7 | VDHIVPRSFMTDNSFD | Mut 2.1 |
| 8 | VDHILPRSYMKDDSFD | Mut 2.2 |
| 9 | VDHIIPRSFLRNDSLD | Mut 2.4 |
| 10 | VDHVIPQSFMTDDSIE | Mut 2.10 |
| 11 | LEKQGFVKRQLMETR | Mut 3.8 |
| 12 | LDEQRWIKRQLVETQ | Mut 3.9 |
| 13 | LDEARWVKRQLMETR | Mut 3.10 |
| 14 | RENQTTQKGQKNSRERMKRIEEGIKELGSQILKEHPVENTQLQNEKL | SpCas9 HNH |
| | YLYYLQNGRDMYVDQELDINRLSDYDVDHIVPQSFLKDDSIDNKVL | domain |
| | TRSDKNRGKSDNVPSEEVVKKMKNYWRQLLNAKLITQRKFDNLTK | |
| | AERGGLSELDKAGFIKRQLVETR | |

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a treatment” includes a single treatment, as well as two or more treatments; and so forth.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In the context of this specification, the term “about” is understood to refer to a range of numbers that a person of skill in the art would consider equivalent to the recited value in the context of achieving the same function or result.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

The “clustered regularly interspaced short palindromic repeat” (CRISPR)/“CRISPR-associated protein” (Cas) system (CRISPR/Cas system) evolved in bacteria and archaea as an adaptive immune system to defend against viral attack. Upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence.

The terms “guide RNA” or “gRNA” refer to a RNA sequence that is complementary to a target DNA and directs a CRISPR endonuclease to the target nucleic acid sequence. gRNA comprises CRISPR RNA (crRNA) and a tracr RNA (tracrRNA). crRNA is a 17-20 nucleotide sequence that is complementary to the target nucleic acid sequence, while the tracrRNA provides a binding scaffold for the endonuclease. crRNA and tracrRNA exist in nature a two separate RNA molecules, which has been adapted for molecular biology techniques using, for example, 2-piece gRNAs such as CRISPR tracer RNAs (cr:tracrRNAs). The skilled person would understand that the term “gRNA” describes all CRISPR guide formats, including two separate RNA molecules or a single RNA molecule. By contrast, the term “sgRNA” will be understood to refer to single RNA molecules combining the crRNA and tracrRNA elements into a single nucleotide sequence.

The mechanisms of CRISPR-mediated genome and gene editing are well known to persons skilled in the art and have been described, for example, by Doudna et al., (2014, Methods in Enzymology, 546).

As described and exemplified herein, the present inventors have generated Cas9 variants (mutants) with improved activity, hence providing for more efficient gene editing. Specifically, the inventors have engineered the HNH-like nuclease domain (also referred to herein as the HNH domain) of Cas9 to increase the rate of gene editing. The HNH-like nuclease domain orchestrates Cas9 cleavage, moving between multiple different positions during the catalytic cycle, and regulates cleavage by the Cas9 RuvC-like nuclease domain. The present disclosure describes Cas9 mutants (also referred to herein as variants, or engineered Cas9 enzymes; and these terms may be used interchangeable herein) containing at least one mutation within one or more of the following regions of the Cas9 HNH-like domain: (1) amino acid positions 765-780 of SEQ ID NO:1; (2) amino acid positions 838-853 of SEQ ID NO:1; and (3) amino acid positions 911-924 of SEQ ID NO:1.

Without wishing to be bound by theory, it is believed that an advantage offered by the Cas9 protein variants described herein is that the low levels of activity and frequent off-target cleavage events observed in CRISPR/Cas systems using wild-type Cas9 enzymes reflects, at least in part, their evolution in bacteria to target rapidly mutating viruses that can infect cells in low numbers.

The inherently low activity of naturally occurring Cas9 enzymes limits their applications where multiple turnover cycles would be advantageous. Again without wishing to be bound by theory, it is suggested that the improved Cas9 variants described herein enable larger numbers of genes to be targeted, e.g. using multiple gRNAs, in cells to elucidate complex genetic interactions, synthetic lethal genes, and the roles of large protein families with overlapping functions. Additionally, these improved variants may be employed in vitro as substitutes for restriction enzymes but with programmable, long and specific target sites that can be modified by substituting different gRNAs.

Furthermore, the improved variants described herein can be used to improve any nickase application where the HNH domain is used to nick a targeted single strand in DNA. Such enhanced nickase activity can be a valuable tool for genome editing. These applications include base editor technologies where nickase-stimulated repair of a deaminated base enables the targeted mutation of DNA with single base resolution. Base editing genome editing technologies use the fusion of deaminase domains to CRISPR enzymes to enable the introduction of point mutations in DNA without generating double strand breaks. The technology typically uses the D10A mutation in the RuvC domain of Cas9 to generate a nickase; which then relies on cleavage by the HNH domain to generate a single stranded nick. Repair of the nicked strand then biases incorporation of deaminated DNA bases and thus the introduction of point mutations into the genome. Two major classes of base editors have been developed: cytidine base editors (CBEs), producing C to T transitions; and adenine base editors (ABEs), producing A to G transitions. Described herein is the ability of Cas9 enzyme variants to enhance base editing, via increased nickase activity of the HNH domain, in the context of ABEs.

Provided herein in embodiments of the present disclosure are Cas9 proteins comprising SEQ ID NO:1 or a sequence at least 80% identical thereto, wherein:
the amino acid residues at positions 765 to 780 are replaced by the amino acid sequence of SEQ ID NO:5 or SEQ ID NO:6;
the amino acid residues at positions 838 to 853 are replaced by the amino acid sequence of SEQ ID NO:7, SEQ ID NO:8, SEQ ID NO:9 or SEQ ID NO:10; and/or
the amino acid residues at positions 911 to 925 are replaced by the amino acid sequence of SEQ ID NO:11, SEQ ID NO:12 or SEQ ID NO:13.

Also provided herein are Cas9 proteins comprising an HNH domain comprising SEQ ID NO:14 or a sequence at least 80% identical thereto, wherein:
the amino acid residues at positions 1 to 16 are replaced by the amino acid sequence of SEQ ID NO:5 or SEQ ID NO:6;
the amino acid residues at positions 74 to 89 are replaced by the amino acid sequence of SEQ ID NO:7, SEQ ID NO:8, SEQ ID NO:9 or SEQ ID NO:10; and/or
the amino acid residues at positions 147 to 161 are replaced by the amino acid sequence of SEQ ID NO:11, SEQ ID NO:12 or SEQ ID NO:13.

In exemplary embodiments a Cas9 protein of the present disclosure comprises the amino acid sequence of SEQ ID NO:5 or SEQ ID NO:6 and the amino acid sequence of SEQ ID NO:7, SEQ ID NO:8, SEQ ID NO:9 or SEQ ID NO:10, at positions 765 to 780 and positions 838 to 853, respectively, of SEQ ID NO:1, or at positions 1 to 16 and positions 74 to 89, respectively, of SEQ ID NO:14.

In exemplary embodiments a Cas9 protein of the present disclosure comprises the amino acid sequence of SEQ ID NO:5 or SEQ ID NO:6 and the amino acid sequence of SEQ ID NO:11, SEQ ID NO:12, or SEQ ID NO:13, at positions 765 to 780 and positions 911 to 925, respectively, of SEQ ID NO:1, or at positions 1 to 16 and positions 147 to 161, respectively, of SEQ ID NO:14.

In a particular exemplary embodiment, the Cas9 protein comprises the amino acid sequence of SEQ ID NO:6 and the amino acid sequence of SEQ ID NO:11, at positions 765 to 780 and positions 911 to 925, respectively, of SEQ ID NO:1, or at positions 1 to 16 and positions 147 to 161, respectively, of SEQ ID NO:14. In a further particular exemplary embodiment, the Cas9 protein comprises the amino acid sequence of SEQ ID NO:6 and the amino acid sequence of SEQ ID NO:12, at positions 765 to 780 and positions 911 to 925, respectively, of SEQ ID NO:1, or at positions 1 to 16 and positions 147 to 161, respectively, of SEQ ID NO:14.

In exemplary embodiments a Cas9 protein of the present disclosure comprises the amino acid sequence of SEQ ID NO:7, SEQ ID NO:8, SEQ ID NO:9 or SEQ ID NO:10 and the amino acid sequence of SEQ ID NO:11, SEQ ID NO:12 or SEQ ID NO:13, at positions 838 to 853 and positions 911 to 925, respectively, of SEQ ID NO:1, or at positions 74 to 89 and positions 147 to 161, respectively, of SEQ ID NO:14.

In exemplary embodiments a Cas9 protein of the present disclosure comprises the amino acid sequence of SEQ ID NO:5 or SEQ ID NO:6, and the amino acid sequence of SEQ ID NO:7, SEQ ID NO:8, SEQ ID NO:9 or SEQ ID NO:10, and the amino acid sequence of SEQ ID NO:11, SEQ ID NO:12 or SEQ ID NO:13, at positions 765 to 780, 838 to 853 and 911 to 925, respectively, of SEQ ID NO:1, or at positions 1 to 16, 74 to 89 and 147 to 161, respectively, of SEQ ID NO:14.

In a particular exemplary embodiment, the Cas9 protein comprises SEQ ID NO:5, and the amino acid sequence of SEQ ID NO:7, and the amino acid sequence of SEQ ID NO:13, at positions 765 to 780, 838 to 853 and 911 to 925, respectively, of SEQ ID NO:1, or at positions 1 to 16, 74 to 89 and 147 to 161, respectively, of SEQ ID NO:14. In a further particular exemplary embodiment, the Cas9 protein comprises SEQ ID NO:6, and the amino acid sequence of SEQ ID NO:7, and the amino acid sequence of SEQ ID NO:11, at positions 765 to 780, 838 to 853 and 911 to 925, respectively, of SEQ ID NO:1, or at positions 1 to 16, 74 to 89 and 147 to 161, respectively, of SEQ ID NO:14. . In a further particular exemplary embodiment, the Cas9 protein comprises SEQ ID NO:6, and the amino acid sequence of SEQ ID NO:8, and the amino acid sequence of SEQ ID NO:11, at positions 765 to 780, 838 to 853 and 911 to 925, respectively, of SEQ ID NO:1, or at positions 1 to 16, 74 to 89 and 147 to 161, respectively, of SEQ ID NO:14. . In a further particular exemplary embodiment, the Cas9 protein comprises SEQ ID NO:6, and the amino acid sequence of SEQ ID NO:8, and the amino acid sequence of SEQ ID NO:12, at positions 765 to 780, 838 to 853 and 911 to 925, respectively, of SEQ ID NO:1, or at positions 1 to 16, 74 to 89 and 147 to 161, respectively, of SEQ ID NO:14.

For many applications of the CRISPR gene editing system efficiency of Cas9 cleavage may be more important than specificity. Scenarios in which increased activity of Cas9, such as provided by mutants described herein, may be beneficial include, for example, applications where multiple genes may need to be targeted simultaneously (such as oncogenes to halt cancer cell growth), where multiple cleavage events would be required, such as in vitro applications using Cas9 analogous to a restriction enzyme (Karvelis et al., 2013, Biochem Soc Trans 41:1401-1406), or in situations where cleavage efficiency might be limiting. Hyperactive Cas9 mutants described herein provide new tools to address such scenarios inter alia. Furthermore, the ability of Cas9 mutants described herein to introduce more extensive deletions and complex repair scars from multiple edits may be useful to more effectively knockout genes or to provide diverse signatures for cellular recording and lineage tracing (Farzadfard et al., 2018, Science 361:870-875). The skilled addressee will appreciate that the applications of the Cas9 mutants described herein are not limited to those described above.

For applications in which a hyperactive Cas9 enzyme may be beneficial particular embodiments of the present disclosure provide, for example, a Cas9 protein comprising the amino acid sequence of SEQ ID NO:1 or a sequence at least about 80% identical thereto, wherein the amino acid residues at positions 838 to 853 are replaced by the amino acid sequence of SEQ ID NO:8. For applications in which a hyperactive Cas9 enzyme may be beneficial, particular embodiments of the present disclosure provide, for example, a Cas9 protein comprising an HNH domain comprising the amino acid sequence of SEQ ID NO:14 or a sequence at least about 80% identical thereto, wherein the amino acid residues at positions 74 to 89 are replaced by the amino acid sequence of SEQ ID NO:8.

Typically, the proteins provided in accordance with the disclosure are isolated proteins. As used herein, “isolated” with reference to a protein, means that the protein is substantially free of cellular material or other contaminating proteins from the cells from which the protein is derived (and thus altered from its natural state), or substantially free from chemical precursors or other chemicals when chemically synthesized, and thus altered from its natural state.

The terms “protein”, “peptide” and “polypeptide” may be used interchangeably herein to refer to a polymer of amino acid residues linked together by peptide (amide) bonds. The terms refer to a protein, peptide, or polypeptide of any size, structure or function.

The terms “Cas9” and “Cas9 protein” as used herein refer to an RNA-guided nuclease comprising a Cas9 protein, or a fragment thereof. Cas9 nuclease sequences would be known to persons skilled in the art, illustrative examples of which are described by, for example Ferretti et al. (2001, Proceedings of the National Academy of Science U.S.A., 98: 4658-4663), Deltcheva et al. (2011, Nature, 471: 602-607), and Jinek et al. (2012, Science, 337: 816-821).

In particular embodiments the Cas9 proteins of the present disclosure are derived from Streptococcus pyogenes Cas9 (SpCas9). As used herein the term “derived” means that the amino acid sequence of the protein of the present disclosure substantially corresponds to, originates from, or otherwise shares significant sequence homology with the sequence of SpCas9. Those skilled in the art will understand that by being “derived” from a naturally occurring or native Cas9 sequence, the sequence in a protein of the present disclosure need not be physically constructed or generated from the naturally occurring or native Cas9 sequence, but may be recombinantly generated or otherwise synthesised such that the sequence is “derived” from the naturally occurring or native Cas9 sequence in that it shares sequence homology and function with the naturally occurring or native sequence.

The terms “wild-type”, “native” and “naturally occurring” are used interchangeably herein to refer to a gene or gene product that has the characteristics of that gene or gene product when isolated from a naturally occurring source. A wild type, native or naturally occurring gene or gene product is that which is most frequently observed in a population and is thus arbitrarily designed the “normal” or “wild-type” form of the gene or gene product.

In accordance with the present disclosure, the HNH domain may be derived from SpCas9 and may comprise, absent the replacement residues defined herein, the amino acid sequence of SEQ ID NO:14 or an amino acid sequence which is at least 80% identical to the amino acid sequence of SEQ ID NO: 14. Accordingly, the sequence may be at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 14.

Similarly, in accordance with the present disclosure, the Cas9 protein may be derived from SpCas9 and may comprise, absent the replacement residues defined herein, the amino acid sequence of SEQ ID NO:1 or an amino acid sequence which is at least 80% identical to the amino acid sequence of SEQ ID NO: 1. Accordingly, the sequence may be at least 80%, at least 81%, at least 82%, at least 83%, at least 84%, at least 85%, at least 86%, at least 87%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 1.

The term “sequence identity” as used herein in the context of amino acid sequences refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue occurs in both sequences to yield the number of matched positions, dividing the number of matched positions by the total number of positions in the window of comparison (i.e., the window size), and multiplying the result by 100 to yield the percentage of sequence identity.

Methods for the determination of sequence identity would be known to persons skilled in the art, illustrative examples of which include computerized implementations of algorithms (BLAST, GAP, BESTFIT, FASTA, and TFASTA in the Wisconsin Genetics Software Package Release 7.0, Genetics Computer Group, 575 Science Drive Madison, WI, USA). Exemplary reference may be made to the BLAST family of programs as, for example, disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

In an exemplary embodiment, a Cas9 protein of the present disclosure comprises the amino acid sequence of SEQ ID NO:1 or sequence at least 80% identical thereto, wherein: the amino acid residues at positions 765 to 780 are replaced by the amino acid sequence of SEQ ID NO:5 or SEQ ID NO:6; the amino acid residues at positions 838 to 853 are replaced by the amino acid sequence of SEQ ID NO:7, SEQ ID NO:8, SEQ ID NO:9 or SEQ ID NO:10; and/or the amino acid residues at positions 911 to 925 are replaced by the amino acid sequence of SEQ ID NO:11, SEQ ID NO:12 or SEQ ID NO:13.

In an exemplary embodiment, the amino acid residues at positions 765 to 780 are replaced by the amino acid sequence of SEQ ID NO:5, the amino acid residues at positions 838 to 853 are replaced by the amino acid sequence of SEQ ID NO:7, and the amino acid residues at positions 911 to 925 are replaced by the amino acid sequence of SEQ ID NO:13. In another exemplary embodiment, the amino acid residues at positions 765 to 780 are replaced by the amino acid sequence of SEQ ID NO:6, the amino acid residues at positions 838 to 853 are replaced by the amino acid sequence of SEQ ID NO:7, and the amino acid residues at positions 911 to 925 are replaced by the amino acid sequence of SEQ ID NO:11. In another exemplary embodiment, the amino acid residues at positions 765 to 780 are replaced by the amino acid sequence of SEQ ID NO:6, the amino acid residues at positions 838 to 853 are replaced by the amino acid sequence of SEQ ID NO:8, and the amino acid residues at positions 911 to 925 are replaced by the amino acid sequence of SEQ ID NO:11. In another exemplary embodiment, the amino acid residues at positions 765 to 780 are replaced by the amino acid sequence of SEQ ID NO:6, the amino acid residues at positions 838 to 853 are replaced by the amino acid sequence of SEQ ID NO:8, and the amino acid residues at positions 911 to 925 are replaced by the amino acid sequence of SEQ ID NO:12.

As described herein, the Cas9 protein may be derived from the Cas9 protein of Streptococcus pyogenes.

Also provided herein is an isolated Cas9 protein comprising an HNH domain comprising the amino acid sequence of SEQ ID NO:14 or a sequence at least about 80% identical thereto, wherein: the amino acid residues at positions 1 to 16 are replaced by the amino acid sequence of SEQ ID NO:5 or SEQ ID NO:6; the amino acid residues at positions 74 to 89 are replaced by the amino acid sequence of SEQ ID NO:7, SEQ ID NO:8, SEQ ID NO:9 or SEQ ID NO:10; and/or the amino acid residues at positions 147 to 161 are replaced by the amino acid sequence of SEQ ID NO:11, SEQ ID NO:12 or SEQ ID NO:13.

The present disclosure also contemplates conservatively substituted variants of the Cas9 proteins described herein. A conservative substitution refers to an amino acid substitution that does not significantly affect or alter the binding or catalytic properties of the protein. Those skilled in the art will recognize that amino acid residues may be replaced with other amino acid residue having a side chain with similar properties, such as a similar charge. Families of amino acid residues having similar side chains have been defined in the art (see, for example, Lehninger, A. L., 1975, Biochemistry, 2^{nd} Edition, Worth Publishers (NY) and Zubay, G., 1988, Biochemistry, 2^{nd} Edition, Macmillan Publishing (NY)). These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). The skilled person will appreciate that it is reasonable to expect that replacement of an amino acid with a structurally related amino acid within the same family as defined above will not have a significant effect on the properties of the resulting variant polypeptide.

Thus, a conservatively substituted variant of a Cas9 protein described herein is a variant substantially homologous to the protein of which it is a variant but in which the sequence includes one or more conservative substitutions. Such substitutions can be introduced into a protein by standard techniques known in the art, such as site-directed mutagenesis and PCR-mediated mutagenesis. The resultant variants can be tested for retained function by any method known to those skilled in the art without undue experimentation.

The present disclosure contemplates full-length Cas9 proteins as well as catalytically active fragments thereof.

A Cas9 protein of the present disclosure may further comprise one or more additional domains or moieties. For example, the protein may comprise one or more deaminase domains, cell recognition or targeting domains, nuclear localization signals (NLS), and/or antibiotic selection domains (e.g., blasticidin-S-deaminase).

Embodiments of the disclosure contemplate derivatives of the proteins disclosed herein. As used herein the term “derivative” is intended to encompass chemical modification to a protein or one or more amino acid residues of a protein, including chemical modification in vitro, for example, by introducing a group in a side chain in one or more positions of a peptide, such as a nitro group in a tyrosine residue or iodine in a tyrosine residue, by conversion of a free carboxylic group to an ester group or to an amide group, by converting an amino group to an amide by acylation, by acylating a hydroxy group rendering an ester, by alkylation of a primary amine rendering a secondary amine, or linkage of a hydrophilic moiety to an amino acid side chain Other derivatives may be obtained by oxidation or reduction of the side-chains of the amino acid residues in the protein. Modification of an amino acid may also include derivation of an amino acid by the addition and/or removal of chemical groups to/from the amino acid, and may include substitution of an amino acid with an amino acid analog (e.g., a phosphorylated or glycosylated amino acid) or a non-naturally occurring amino acid such as a N-alkylated amino acid (e.g., N-methyl amino acid), D-amino acid, β-amino acid or γ-amino acid.

The proteins of the present disclosure may be produced using any method known in the art, including standard techniques of recombinant DNA and molecular biology that are well known to those skilled in the art. Guidance may be obtained, for example, from standard texts such as Sambrook et al., Molecular Cloning: A Laboratory Manual, Cold Spring Harbor, New York, 1989 and Ausubel et al., Current Protocols in Molecular Biology, Greene Publ. Assoc. and Wiley-Intersciences, 1992. The skilled addressee will appreciate that the present disclosure is not limited by the method of production or purification used and any other method may be used to produce Cas9 proteins in accordance with the present disclosure.

The present disclosure also provides isolated polynucleotides encoding the Cas9 proteins described herein. As used herein the terms “polynucleotide”, “nucleotide sequence” or “nucleic acid sequence” mean a single- or double-stranded polymer of deoxyribonucleotide, ribonucleotide bases or known analogues or natural nucleotides, or mixtures thereof, and include coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

As used herein, the terms “encode,” “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The present disclosure also provides delivery vehicles comprising a polynucleotide sequence(s) encoding a Cas9 protein described herein. In some embodiments, nucleic acid molecules are packaged into or on the surface of delivery vehicles for delivery to cells. Delivery vehicles contemplated include, but are not limited to, nanospheres, liposomes, ribonucleoproteins, positively charged peptides, small molecule RNA-conjugates, quantum dots, nanoparticles, polyethylene glycol particles, hydrogels, and micelles. As described in the art, a variety of targeting moieties can be used to enhance the preferential interaction of such vehicles with desired cell types or locations.

Polynucleotide sequences encoding Cas9 proteins described herein can be incorporated into viral or non-viral vectors. Typically the polynucleotide sequence(s) is operably linked to a promoter to allow for expression of the fusion peptide or components thereof. In some embodiments, the vector further comprises a polynucleotide encoding a gRNA.

The vectors can be episomal vectors (i.e., that do not integrate into the genome of a host cell), or can be vectors that integrate into a host cell genome. Vectors may be replication competent or replication-deficient. Exemplary vectors include, but are not limited to, plasmids, cosmids, and viral vectors, such as adeno-associated virus (AAV) vectors, lentiviral, retroviral, adenoviral, herpesviral, parvoviral and hepatitis viral vectors. The choice and design of an appropriate vector is within the ability and discretion of one of ordinary skill in the art. Preferably, however, the vector is suitable for use in gene therapy.

Vectors suitable for use in gene therapy would be known to persons skilled in the art, illustrative examples of which include viral vectors derived from adenovirus, adeno-associated virus (AAV), herpes simplex virus (HSV), retrovirus, lentivirus, self-amplifying single-strand RNA (ssRNA) viruses such as alphavirus (e.g., Semliki Forest virus, Sindbis virus, Venezuelan equine encephalitis, M1), and flavivirus (e.g., Kunjin virus, West Nile virus, Dengue virus), rhabdovirus (e.g., rabies, vesicular stomatitis virus), measles virus, Newcastle Disease virus (NDV) and poxivirus as described by, for example, Lundstrom (2019, Diseases, 6: 42).

In an exemplary embodiment, the vector is an adeno-associated virus (AAV) vector. Exemplary AAV vectors include, without limitation, those derived from serotypes AAV1, AAV2, AAV3, AAV4, AAV5, AAV6, AAV7, AAV8, AAV9, AAV10, AAV11, AAV12 or AAV13, or using synthetic or modified AAV capsid proteins such as those optimized for efficient in vivo transduction. A recombinant AAV vector describes replication-defective virus that includes an AAV capsid shell encapsidating an AAV genome. Typically, one or more of the wild-type AAV genes have been deleted from the genome in whole or part, preferably the rep and/or cap genes.

The present disclosure also provides non-viral methods of delivery of the Cas9 proteins described herein. Suitable non-viral delivery methods will be known to persons skilled in the art, illustrative examples of which include using lipids, lipid-like materials or polymeric materials, as described, for example, by Rui et al. (2019, Trends in Biotechnology, 37(3): 281-293), and nanoparticles, as described, for example, by Nguyen et al. (2020, Nature Biotechnology, 38: 44-49).

The Cas9 proteins of the present disclosure find application in any CRISPR/Cas9 system for genome or gene editing, for example for introducing mutations, deletions, alterations, integrations, gene correction, gene replacement, gene tagging, transgene insertion, nucleotide deletion, gene disruption, and/or translocations and/or gene mutation. The process of integrating non-native nucleic acid into genomic DNA is an example of genome editing. Applications and uses of the CRISPR/Cas9 system will be well known to those skilled in the art; for example international patent application publication number WO 2013/176772 provides numerous examples and applications of the CRISPR/Cas system for site-specific gene editing.

Accordingly, provided herein is a complex comprising a Cas9 protein as described herein and a guide RNA (gRNA) bound to the HNH domain of the Cas9 protein. Also provided is a method for editing the genome of a cell, comprising providing to the cell a Cas9 protein as described herein or nucleic acid encoding said Cas9 protein and a gRNA complementary to a target sequence within a target genomic locus in the cell, or nucleic acid encoding the gRNA.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### General Methods

### Plasmid Construction

SpCas9 was codon optimized using Gene Designer software (ATUM), synthesized by IDT in 4 gBlocks and assembled using Gibson assembly in the pJ201 plasmid. The Cas9 ORF was flanked by BamHI and NotI restriction sites for sub-cloning into the yeast expression plasmids pCM251 and pCM252. Three regions of the HNH domain were selected for in silico mutagenesis and structural repair, which were flanked by SpeI-BsaI, BsmBI-SacII and XbaI and StuI restriction sites, respectively. Each region containing the designed mutations was designed in Gene Designer and synthesized by Twist. Each mutant region was either individually cloned into Cas9 or simultaneously as combinations. The mutant region of the HNH domain of Mut 1.5-3.8 (see Example 3) was codon optimized for mammalian cells and subcloned into the mammalian expression vector pD1311-AD (ATUM) for double strand break editing or pCMV_ABEmax_P2A_GFP (see Koblan et al., 2018, Nat Biotechnol 36:843-846). The pRS426-Can1 gRNA plasmid²⁵ was obtained from Addgene (#43803) and two separate gRNAs targeting ADE2 and HIS3 were synthesized by IDT. The CAN1 gRNA was swapped with either ADE2 or HIS3 gRNA using the flanking restriction enzymes NheI and MluI. The Cas9 inhibitors AcrIIA2 and AcrIIA4 fused with a P2A peptide and flanked by the CUP1 promoter and PGI1 terminator was ordered as a gBlock from IDT. The expression cassette was flanked by KpnI and MluI for cloning into the pRS426 gRNA plasmid.

### Yeast Transformation

A single colony of S. cerevisiae strain BY4738 (MATα trp1Δ63 ura3Δ0) was used to inoculate 2 ml YPAD and grown overnight at 30° C. Cells were pelleted at 3200 rpm for 2 min, resuspended in 50 ml YPAD in a baffled task and incubated for 3 h at 30° C. Cells were spun down at 3400×g for 2 min and washed in 50 mL 1×TE. The pellet was resuspended in 2 mL 100 mM lithium acetate/0.5×TE and incubated at room temperature for 10 minutes. An aliquot of 100 μl of cells was gently mixed with 1 μg of DNA for transformation and 100 μg of denatured salmon sperm DNA. To this, 700 μl of 40% PEG 3500/100 mM Lithium Acetate/1×TE was added and carefully mixed and incubated in a water bath for 30 minutes at 30° C. The cells were heat shocked at 42° C. for 7 min in a water bath after addition of 88 μl DMSO. Cells were collected by centrifugation and washed in 1 mL 1×TE. The pellet was resuspended in 100 μl 1×TE and plated out on SC-T-U and incubated at 30° C. for 2-3 days.

### Dotting

A single colony was grown overnight in 10 ml of SC-T-U media at 30° C. Yeast cultures were standardized to one OD₆₀₀ in 1×TE and three serial 1/10 dilutions were made in 1×TE buffer. Of each dilution 5 μl were plated out on selective media (SC) with the appropriate amino acids lacking and supplemented with anhydrotetracycline (ATC) were indicated. Plates were grown for 2-3 days at 30° C.

### Quantitative Survival Assay

A single colony was grown overnight in 10 ml of SC-T-U media at 30° C. Cells were standardized to one OD₆₀₀ and diluted to 2.8×10⁻³ in 1×TE. Of each sample 100 μl were plated out on selective media with or without anhydrotetracycline lacking the appropriate auxotrophic nutrients and grown for 2 days at 30° C.

### Culturing and Transfection of Mammalian Cells and Amplicon Sequencing

HEK293T cells were cultured at 37° C. in humidified 95% air/5% CO2 in Dulbecco's modified Eagle's (DMEM; Gibco, Life Technologies) containing glucose (4.5 g/L), fetal bovine serum (FBS; 10%), 1 mM sodium pyruvate and 2 mM glutamine. Cells were seeded at 60% confluence in 24-well plates, allowed to attach overnight and were transfected with 500 ng (158 ng/cm²) of plasmid DNA. Transfections were performed using a 1:1 ratio of FuGENE HD (Promega) and Lipofectamine LTX (Invitrogen) in Opti-MEM media (Gibco, Life Technologies). 72 h after transfection the cells were trypsinized and the cell pellets lysed for DNA extraction using the KAPA Express Extract Kit, according to the manufacturer's instructions (Sigma-Aldrich). Amplicons were generated using primers flanking the gRNA and incorporating Illumina adaptor sequences (Supplementary Table 2). Amplicons were sequenced on an Illumina MiSeq using 250 bp paired end chemistry by the Australian Genomics Research Facility (AGRF), Perth, Western Australia.

### Next Generation Sequencing Analyses

Sequenced reads were trimmed with TrimGalore27 (v0.6.6) using cutadapt28 (v1.18) and fastqc29 (v0.11.9) (--paired --nextera --fastqc). Trimmed reads were merged with FLASH30 (v1.2.11) (--min-overlap 10 --max-overlap 250). Initially merged reads were aligned to amplicon sequences with bowtie2. Long and complex deletions and insertions that matched the ends of the amplicon were soft-clipped by bowtie2. To evaluate long deletions the merged reads were aligned against their respective amplicon sequences with BLAT31 (v37x1) (-minScore=0 -stepSize=1 -out=psl). The resultant .psl file was converted to SAM/BAM format with the uncle_psl.py32. The resulting BAM files were parsed with command-line tools based on the number of alphabetic characters in the CIGAR sequence (termed CIGAR 401 complexity herein). Since these characters represent specific alignment characteristics (match, insertion, deletion or soft-clipping) and are paired with a number describing their length, the inventors used this information to determine the lengths and locations of deletion and insertion events for all alignments. Alignments that contained soft clipped sequences, or with a CIGAR complexity of 7 or above, were excluded. All configurations of alignment up to a CIGAR complexity of 6 and the most simple of complexity 7 (MIDMIDM) were collated and summarized.

### Example 1—Design of a Yeast-Based Cas9 Efficacy Screen

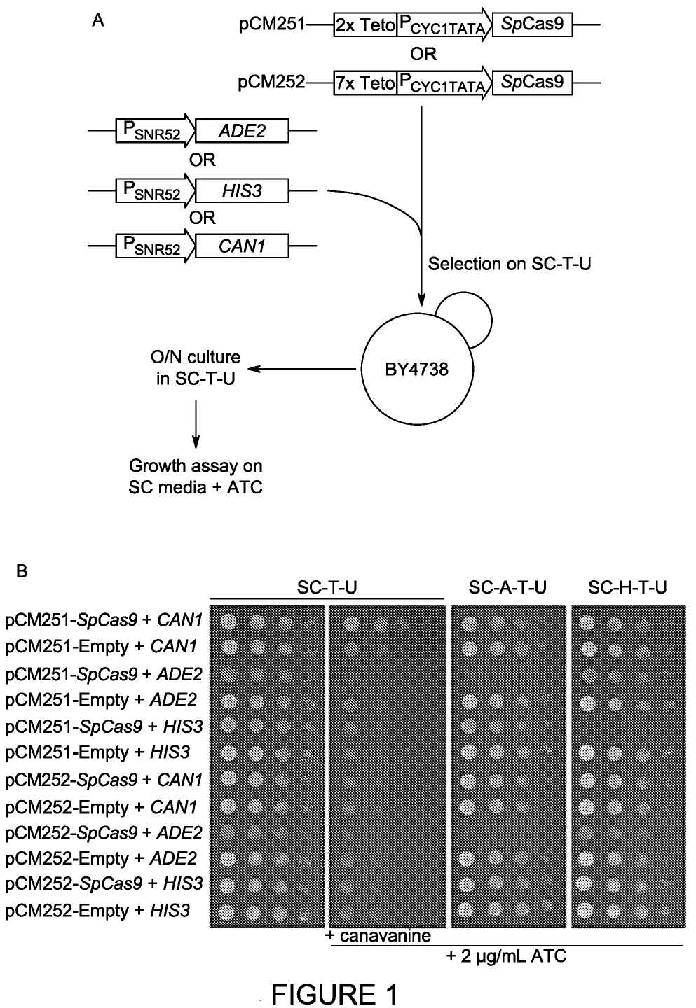

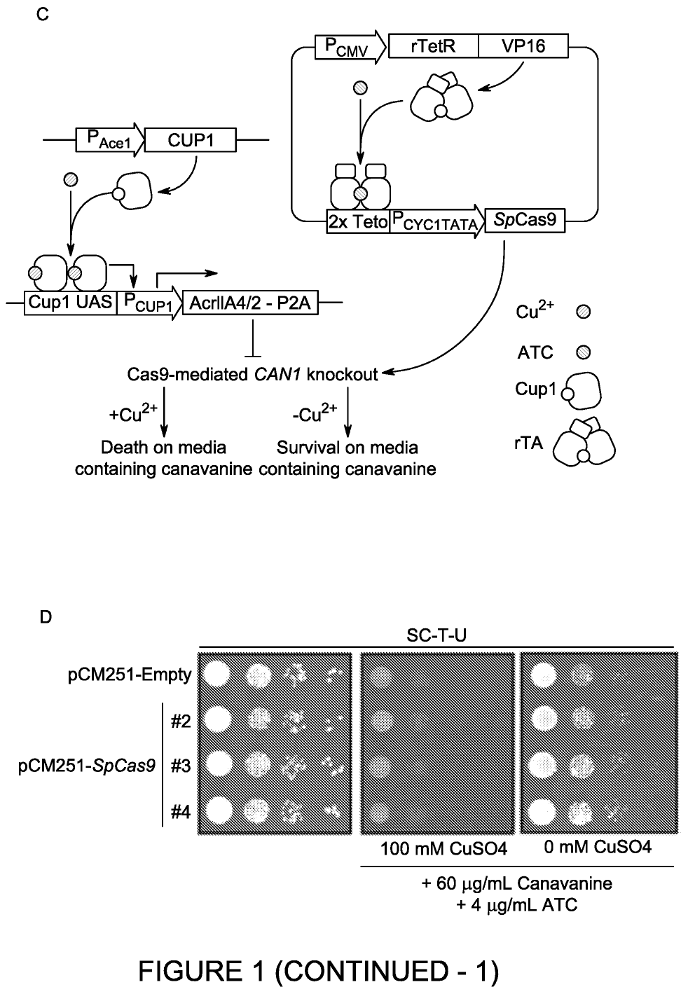

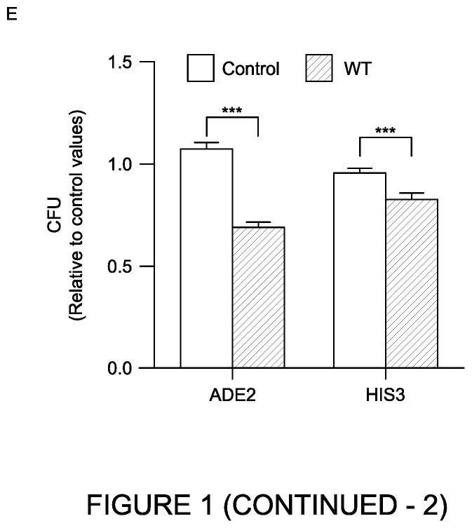

The inventors designed a yeast-based reporter system consisting of a gRNA vector and a tetracycline inducible Cas9 expression plasmid to compare the enzymatic activities of mutagenized Cas9 enzymes to wild-type SpCas9 (FIG. 1A). Cas9 was targeted towards the auxotrophic marker genes; ADE2, HIS3 as well as CAN1, an arginine permease, and analysed using a dotting-based survival assay in the Saccharomyces cerevisiae strain BY4738 (Brachmann et al., 1998, Yeast 14, 115-132) (FIG. 1B). It was hypothesized that a knockout of the genes ADE2 and HIS3 could lead to suppressed growth of yeast on synthetic media lacking histidine or adenine when compared to a negative control. In contrast, knockout of CAN1 would lead to an increased growth on media supplemented with canavanine. Initially, the inventors used two different yeast expression plasmids, namely pCM251 and pCM252, which differ only in their number of tetracycline-responsive operator elements, 2 and 7 respectively. Induction of Cas9 was found to be less consistent when using pCM252, therefore all experiments done hereafter were performed with pCM251.

While this system proved to be highly effective in introducing mutations in all three target genes (FIG. 1B), it was found that yeast containing the ADE2 gRNA were often mutated prior to assays; as yeast would turn red on plasmid maintenance plates and no yeast survived on media without adenine (FIG. 1B). To eliminate this confounding variable, the inventors introduced two known Cas9 inhibitors, AcrIIA2 and AcrIIA4, which have been shown to bind in distinct ways to inhibit the Cas9-gRNA complex (Liu et al., 2019, Mol. Cell 73, 611-620.e3). In addition, mutations in Cas9 that eliminate AcrIIA2's inhibitory effect have no effect on inhibition by AcrIIA4 and vice versa. AcrIIA2 and AcrIIA4 are fused by a self-cleaving peptide (P2A) and expression is controlled with a copper-inducible promoter (CUP1) and cloned on to the gRNA plasmid (FIG. 1C). This was co-transformed with the Cas9 expression plasmid onto plates containing 100 mM copper sulfate. Using this method the inventors were able to inhibit pre-emptive Cas9 activity, as shown using the CAN1 gRNA on plates supplemented with anhydrotetracycline and 100 mM copper (FIG. 1D), while without copper the efficient induction of Cas9 increased survival on plates supplemented with canavanine (FIG. 1D). Therefore, quantification of the enzymatic activity of mutant Cas9 proteins can be efficiently determined in yeast containing the inducible Cas9 inhibitors. The enzymatic activity of wild-type (WT) SpCas9 in the present yeast system was determined using a quantitative survival assay (FIG. 1E) and served as the baseline to compare designed mutants of the present study.

### Example 2—Enhancing the Enzymatic Activity of Cas9 Using Computational Design

To improve the enzymatic activity of Cas9, a computational approach was employed to discover mutants beyond those able to be determined using random mutagenesis. Based on evolutionary conservation active site residues were altered computationally and ranked by their predicted structural energies, based on atomistic simulations using Rosetta design software. To examine the potential for this approach to produce desirable SpCas9 mutants, the inventors focused on the HNH nuclease domain. The HNH nuclease domain is conformationally dynamic, moving between multiple different positions during the Cas9 catalytic cycle and also regulates the cleavage activity of the RuvC-like nuclease domain. Therefore, the inventors hypothesized that this domain would make a good target for mutagenesis to improve Cas9 activity.

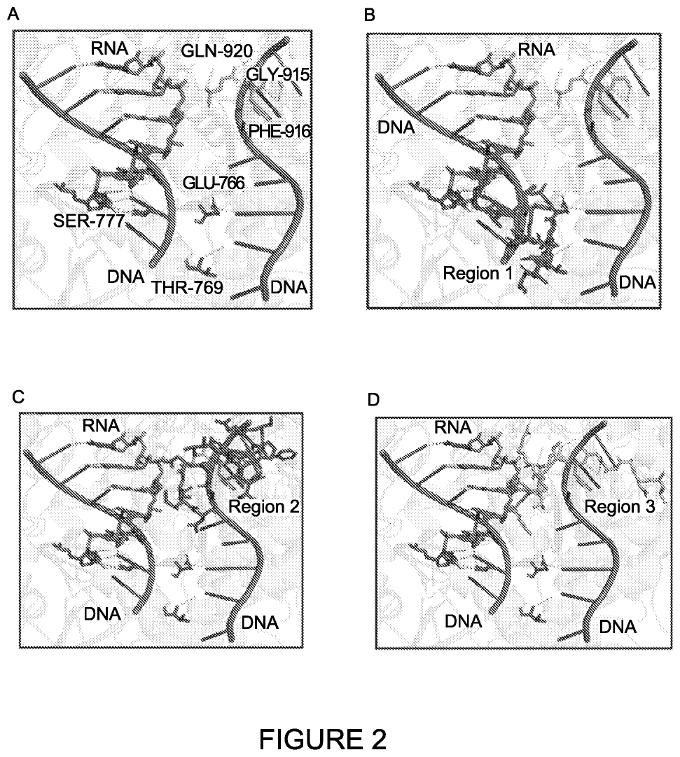

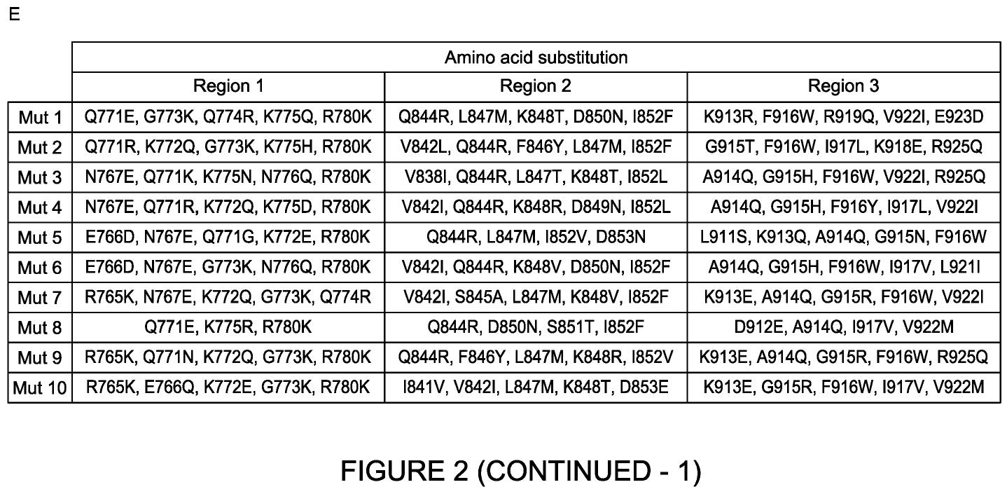

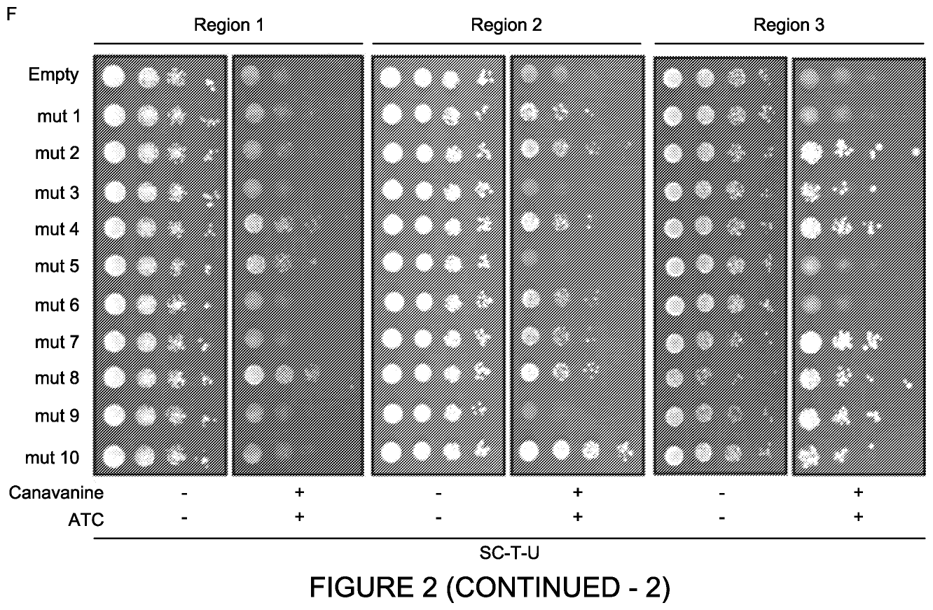

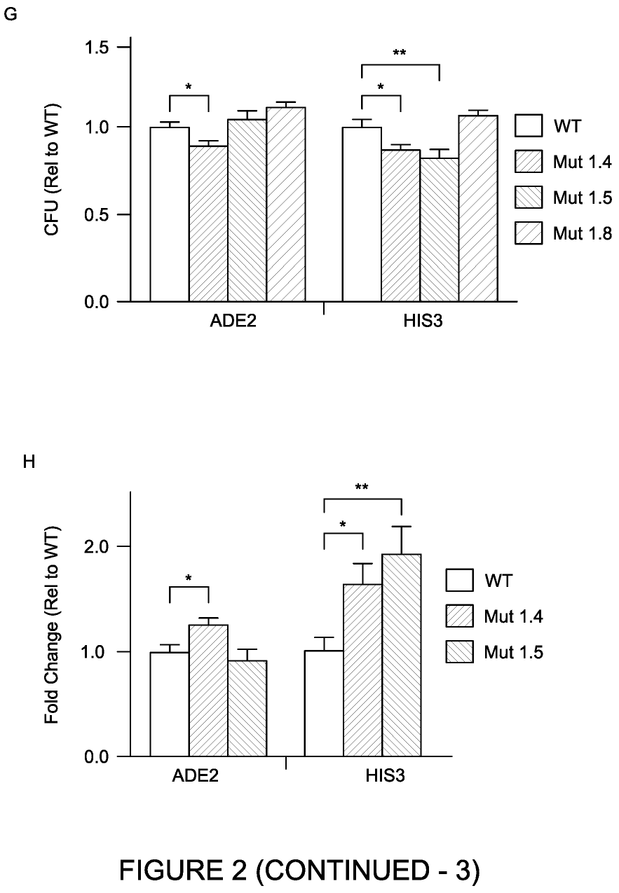

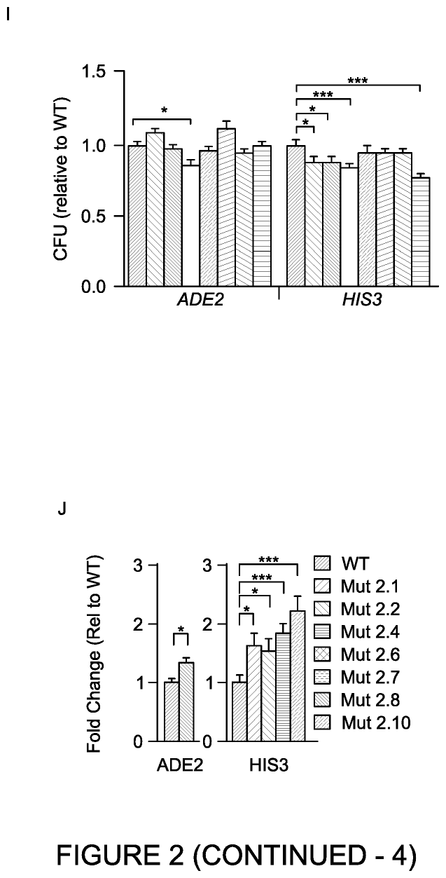

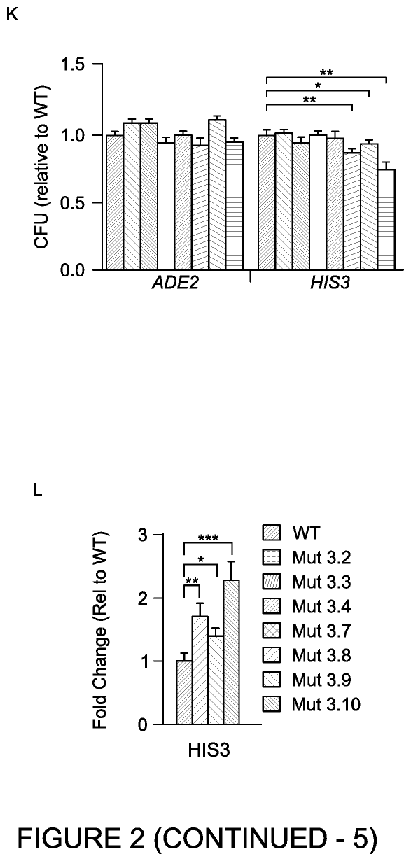

The inventors made three libraries of regions of the SpCas9 HNH nuclease domain. The three regions correspond to: (1) amino acid residues 765 to 780 of SEQ ID NO:1 (SEQ ID NO:2; FIG. 2B); (2) amino acid residues 838 to 853 (SEQ ID NO:3; FIG. 2C); and (3) amino acid residues 911 to 925 (SEQ ID NO:4; FIG. 2D). These regions were chosen as they are either in contact with the target DNA (FIG. 2A) or are required to position active site residues of Cas9 for enzymatic cleavage. For each region the 10 most promising mutants (Mut) (FIG. 2E) based on predicted structural stabilities (in the catalytically active form of Cas9 prior to DNA cleavage), and each containing multiple amino acid substitutions relative to the WT sequence for the relevant region in SpCas9, were chosen and subcloned individually into Cas9 and screened for enzymatic activity (FIG. 2F) as described above in General Methods. Three catalytically active mutants were found for region 1, namely Mut 4, 5 and Mut 8 (designated Mut 1.4, 1.5 and 1.8, respectively). Seven enzymatically active mutants were identified for region 2 (designated Mut 2.1, 2.2, 2.4, 2.6, 2.7, 2.8 and 2.10). Seven enzymatically active mutants were identified for region 3 (designated Mut 3.2, 3.3, 3.4, 3.7, 3.8, 3.9 and 3.10). Enzymatically active mutants were transformed into yeast with the copper inducible Cas9 inhibitors and quantitative survival assays were performed using gRNAs targeting HIS3 to establish the rate of mutagenesis in comparison to the wild type control (SpCas9) (FIG. 2G-L, S2A-D). Of the 17 enzymatically active mutants, several mutants for regions 1, 2 and 3 were found to have a significantly higher activity than WT SpCas9 (FIG. 2G-L; Table 2).

TABLE 2
SpCas9 mutants with changes in region 1, 2 or 3 and displaying most
significantly improved enzymatic activity compared to WT SpCas9

| Mutant | Region of SpCas HNH domain | SEQ ID NO: |
|---|---|---|
| | Region 1^{a} | |
| Mut 1.4 | N767E, Q771R, K772Q, K775D, R780K | 5 |
| Mut 1.5 | E766D, N767E, Q771G, K772E, R780K | 6 |
| | Region 2^{b} | |
| Mut 2.1 | Q844R, L847M, K848T, D850N, 1852F | 7 |
| Mut 2.2 | V842L, Q844R, F846Y, L847M, 1852F | 8 |
| Mut 2.4 | V842I, Q844R, K848R, D849N, I852L | 9 |
| Mut 2.10 | I841V, V842I, L847M, K848T, D853E | 10 |
| | Region 3^{c} | |
| Mut 3.8 | D912E, A914Q, 1917V, V922M | 11 |
| Mut 3.9 | K913E, A914Q, G915R, F916W, R925Q | 12 |
| Mut 3.10 | K913E, G915R, F916W, 1917V, V922M | 13 |

^{a}positions of amino acid changes in each mutant (Mut) are given relative the sequence of HNH domain region 1 of SEQ ID NO:2. Remainder of the sequence of the SpCas9 mutant is SEQ ID NO:1.
^{b}positions of amino acid changes in each mutant (Mut) are given relative the sequence of HNH domain region 2 of SEQ ID NO:3. Remainder of the sequence of the SpCas9 mutant is SEQ ID NO:1.
^{c}positions of amino acid changes in each mutant (Mut) are given relative the sequence of HNH domain region 3 of SEQ ID NO:4. Remainder of the sequence of the SpCas9 mutant is SEQ ID NO:1.

### Example 3—Additive Enzymatic Activities by Combining Mutated Regions of Cas9

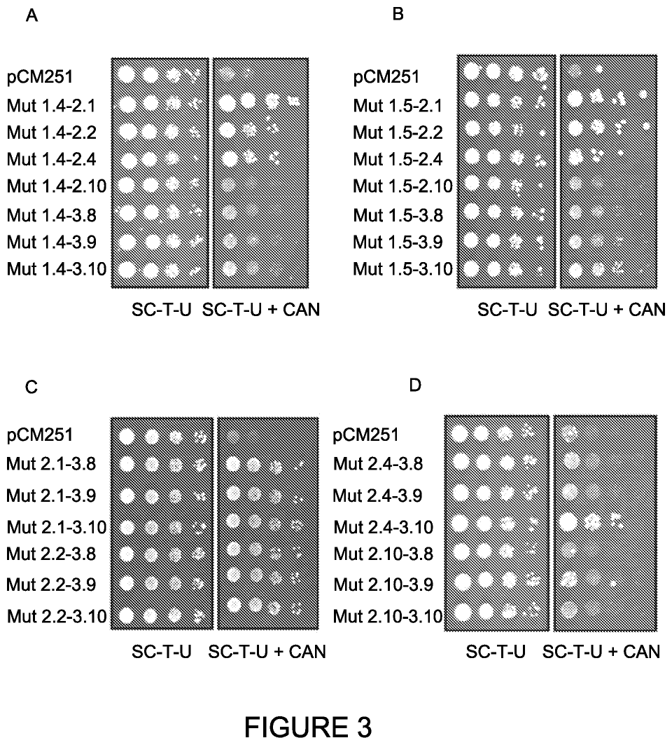

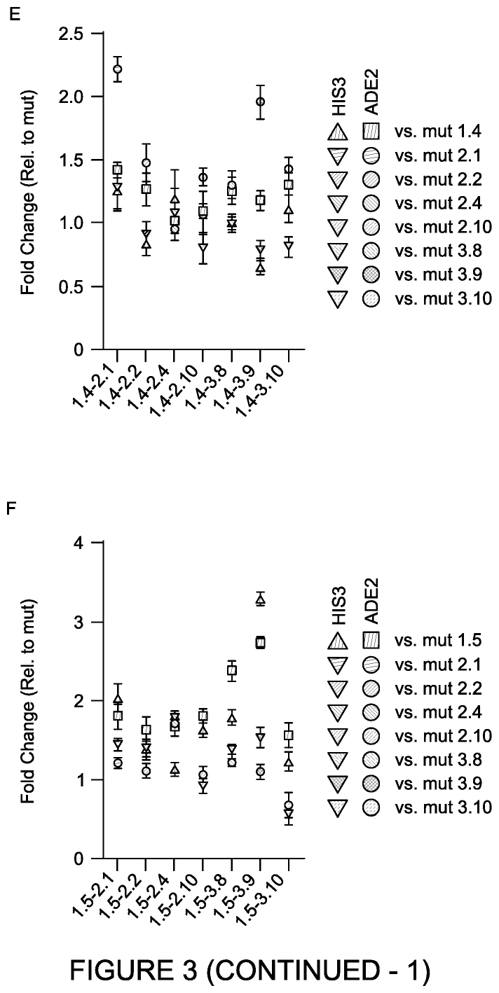

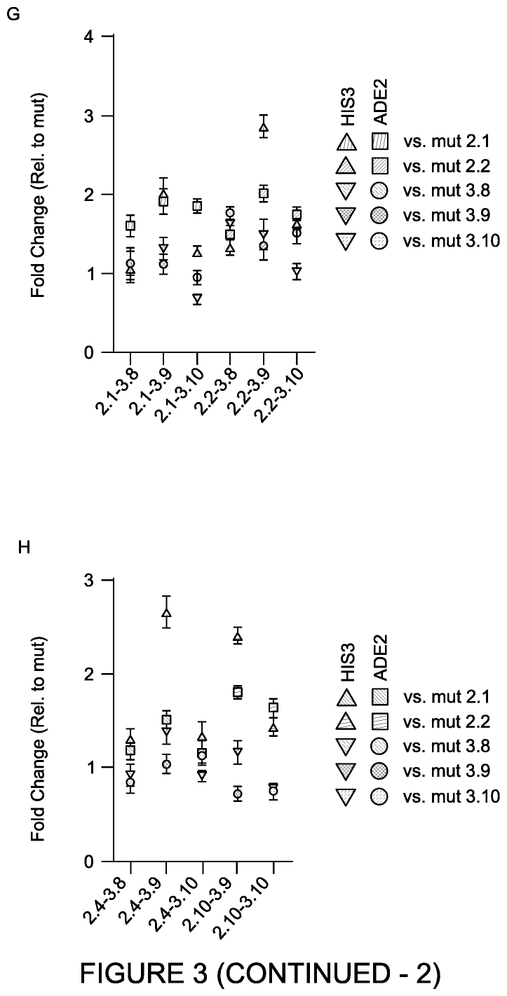

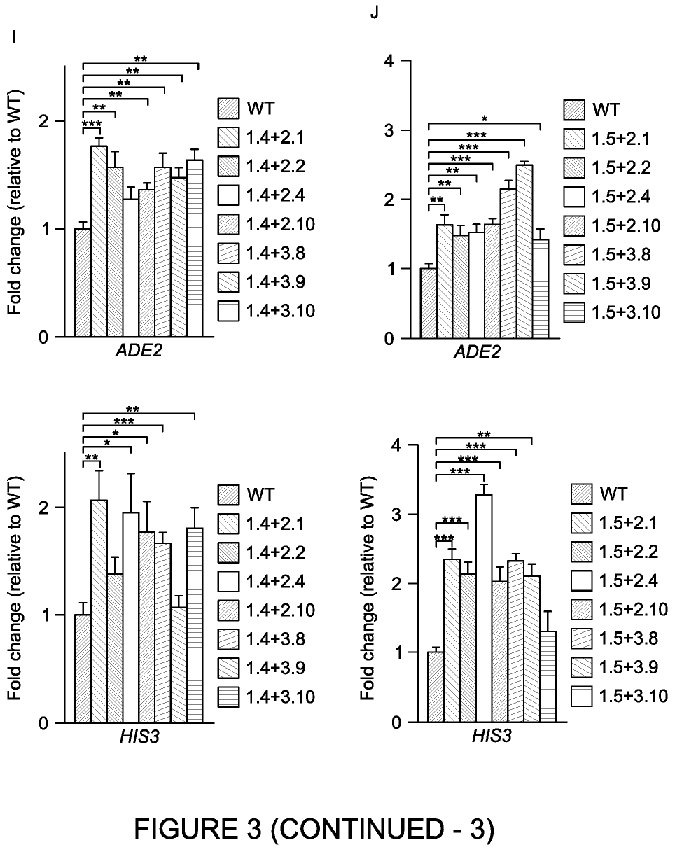

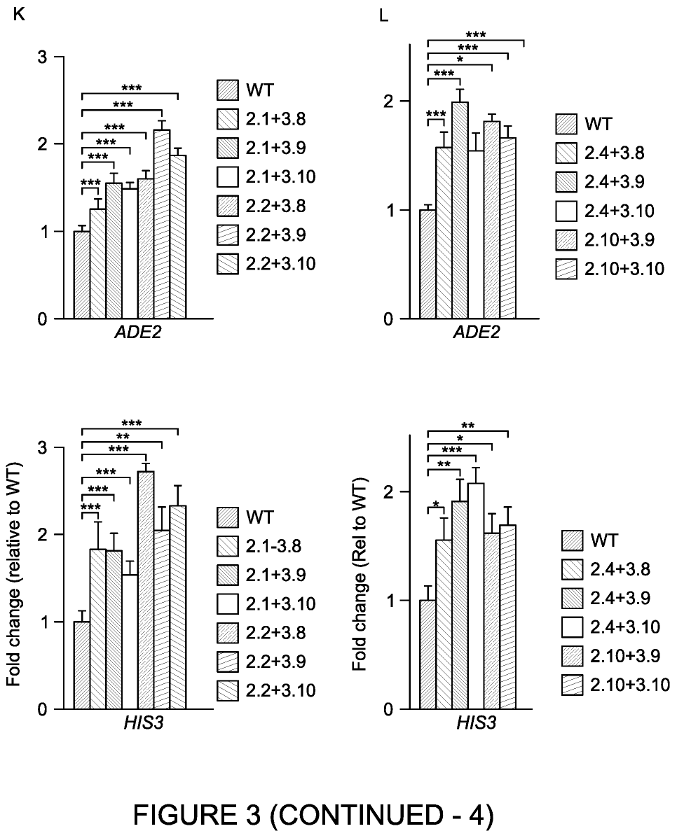

Each of the FuncLib mutants in regions 1,2 and 3 were separately predicted in silico, as such one cannot necessarily assume that these mutants are compatible with each other. However, the inventors hypothesized that there could be potential to further increase the enzymatic activity by combining two or three different mutated regions that showed increased enzymatic activity. Initially, double mutants were made with all possible combinations of the mutant regions that had a significant increase in activity (see Example 2). Enzymatic activity for these combinations of mutants were assessed as described in Example 2 (FIG. 3A-3D). All combinations with exception of Mut 2.10-3.8 (i.e. SpCas9 containing Mut 2.10 in region 2 and Mut 3.8 in region 3) retained their enzymatic activity. Furthermore, a majority of combinations were found to have a significant increase in activity when compared to WT for both gRNAs (FIG. 3I-3L). However, in order to establish that the combinations result in a synergistic increases in activity, the activity of each combination was compared relative to their single mutant counterparts (e.g. Mut 1.4-2.1 compared to both Mut 1.4 and Mut 2.1) (FIG. 3E-3H). The inventors examined the relative improvement of the double mutants compared to their single mutant counterpart and whether the change observed is significant. Most double mutants were found to have either a neutral fold change (FC=˜1, P>0.05) or a positive fold change (FC>1.0, P<0.05). Only mutants 1.4-3.9 and 1.5-3.10 were found to have a negative fold change (FC<1.0, P<0.05) for the HISS gRNA. The double mutants with neutral or positive fold change in enzymatic activity compared to their single mutant counterparts are shown in Table 3.

TABLE 3
SpCas9 double mutants with mutations as described in Table 2 in regions 1 and 2,
regions 1 and 3, or regions 2 and 3, and displaying neutral or positive-fold change in
enzymatic activity compared to single region mutant counterparts

| | | |
|---|---|---|
| Mut 1.4-2.1 | Region 1 Mut 4 and Region 2 Mut 1 | SEQ ID NOs: 5 & 7 |
| Mut 1.4-2.2 | Region 1 Mut 4 and Region 2 Mut 2 | SEQ ID NOs:5 & 8 |
| Mut 1.4-2.4 | Region 1 Mut 4 and Region 2 Mut 4 | SEQ ID NOS:5 & 9 |
| Mut 1.4-2.10 | Region 1 Mut 4 and Region 2 Mut 10 | SEQ ID NOs:5 & 10 |
| Mut 1.4-3.8 | Region 1 Mut 4 and Region 3 Mut 8 | SEQ ID NOs:5 & 11 |
| Mut 1.4-3.10 | Region 1 Mut 4 and Region 3 Mut 10 | SEQ ID NOs:5 & 13 |
| Mut 1.5-2.1 | Region 1 Mut 5 and Region 2 Mut 1 | SEQ ID NOs:6 & 7 |
| Mut 1.5-2.2 | Region 1 Mut 5 and Region 2 Mut 2 | SEQ ID NOS:6 & 8 |
| Mut 1.5-2.4 | Region 1 Mut 5 and Region 2 Mut 4 | SEQ ID NOS:6 & 9 |
| Mut 1.5-2.10 | Region 1 Mut 5 and Region 2 Mut 10 | SEQ ID NOs:6 & 10 |
| Mut 1.5-3.8 | Region 1 Mut 5 and Region 3 Mut 8 | SEQ ID NOs:6 & 11 |
| Mut 1.5-3.9 | Region 1 Mut 5 and Region 3 Mut 9 | SEQ ID NOs:6 & 12 |
| Mut 2.1-3.8 | Region 2 Mut 1 and Region 3 Mut 8 | SEQ ID NOs:7 & 11 |
| Mut 2.1-3.9 | Region 2 Mut 1 and Region 3 Mut 9 | SEQ ID NOs:7 & 12 |
| Mut 2.1-3.10 | Region 2 Mut 1 and Region 3 Mut 10 | SEQ ID NOs:7 & 13 |
| Mut 2.2-3.8 | Region 2 Mut 2 and Region 3 Mut 8 | SEQ ID NOs:8 & 11 |
| Mut 2.2-3.9 | Region 2 Mut 2 and Region 3 Mut 9 | SEQ ID NOs:8 & 12 |
| Mut 2.2-3.10 | Region 2 Mut 2 and Region 3 Mut 10 | SEQ ID NOs:8 & 13 |
| Mut 2.4-3.8 | Region 2 Mut 4 and Region 3 Mut 8 | SEQ ID NOs:9 & 11 |
| Mut 2.4-3.9 | Region 2 Mut 4 and Region 3 Mut 9 | SEQ ID NOs:9 & 12 |
| Mut 2.4-3.10 | Region 2 Mut 4 and Region 3 Mut 10 | SEQ ID NOs:9 & 13 |
| Mut 2.10-3.8 | Region 2 Mut 10 and Region 3 Mut 8 | SEQ ID NOs:10 & 11 |
| Mut 2.10-3.9 | Region 2 Mut 10 and Region 3 Mut 9 | SEQ ID NOs:10 & 12 |
| Mut 2.10-3.10 | Region 2 Mut 10 and Region 3 Mut 10 | SEQ ID NOs:10 & 13 |

Combinations of the Funclib mutants that were significantly increased relative to their single mutant counterparts were used to design triple mutant combinations. These triple mutants are designated by reference to the Mut number for the region 1 mutation followed by the Mut number for the region 2 mutation and the Mut number for the region 3 mutation, such that a triple mutant SpCas9 variant having the Mut 5 mutation in region 1 (Mut 1.5), the Mut 1 mutation in region 2 (Mut 2.1) and the Mut 8 mutation in region 3 (Mut 3.8) is designated Mut 518). Positive mutants for region 1 were combined with positive mutants for both region 2 and 3 to form triple mutants and assessed for catalytic activity. Triple mutants displaying increased activity on ADE2 gRNA are shown in Table 4. Mut 4110 was found to have a fold change of roughly 3.9 in activity on the HIS3 gRNA compared to SpCas9 and a twofold change in activity on the ADE2 gRNA. Significant increased activity was observed for ADE2 and HIS3 gRNAs with all triple mutants based on Mut 1.5. The combined data from the double and triple mutant screening indicates that the enzymatic activity of Cas9 can be further enhanced by combining either two or three computationally designed mutational clusters.

TABLE 4
SpCas9 triple mutants displaying an increase in enzymatic activity compared to
double mutant counterparts

| Mut 4110 | Region 1 Mut 4, Region 2 Mut 1 and Region 3 | SEQ ID NOs: 5, 7 |
|---|---|---|
| | Mut 10 | & 13 |
| Mut 518 | Region 1 Mut 5, Region 2 Mut 1 and Region 3 | SEQ ID NOs: 6, 7 |
| | Mut 8 | & 11 |
| Mut 519 | Region 1 Mut 5, Region 2 Mut 1 and Region 3 | SEQ ID NOs: 6, 7 |
| | Mut 9 | & 12 |
| Mut 528 | Region 1 Mut 5, Region 2 Mut 2 and Region 3 | SEQ ID NOs: 6, 8 |
| | Mut 8 | & 11 |
| Mut 529 | Region 1 Mut 5, Region 2 Mut 2 and Region 3 | SEQ ID NOs: 6, 8 |
| | Mut 9 | & 12 |
| Mut 548 | Region 1 Mut 5, Region 2 Mut 4 and Region 3 | SEQ ID NOs: 6, 9 |
| | Mut 8 | & 11 |
| Mut 549 | Region 1 Mut 5, Region 2 Mut 4 and Region 3 | SEQ ID NOs: 6, 9 |
| | Mut 9 | & 12 |
| Mut 5109 | Region 1 Mut 5, Region 2 Mut 10 and Region | SEQ ID NOs: 6, 10 |
| | 3 Mut 9 | & 12 |

### Example 4—Mutant Activity in Mammalian Cells

One of the most common uses of Cas9 in research is in the creation of knockouts in mammalian cell lines. As such the inventors wanted to verify some of the present mutants in this setting which also allows for the use of commonly used gRNAs that have well-characterized off-target effects. For this, the inventors tested the double mutant showing highest activity for the ADE2 gRNA, Mut 1.5-3.8. This mutant was codon optimized for incorporation into the mammalian system and cloned into the Cas9 expression plasmid pD1311-AD, encoding a GFP-P2A-Cas9 fusion protein while simultaneously expressing a gRNA. On target activity of Cas9 and the Mut 1.5-3.8 mutant was determined for the previously used and well-characterized gRNA targeting the VEGFA (vascular endothelial growth factor A) gene in HEK293T (human embryonic kidney cells). After transfection of HEK293T cells with the Cas9 and VEGFA gRNA expression plasmids the inventors observed a significant increase in editing was observed for the 1.5-3.8 variant when compared to WT Cas9, similar to the observed increased editing in the yeast reporter system.

The inventors subsequently selected 10 active Cas9 mutants (see Table 5) for further testing in mammalian cells.

TABLE 5
SpCas9 mutants with mutations as described in Tables 2 and 3, selected for activity
testing in mammalian cells

| | | |
|---|---|---|
| Mut 1.4 | Region 1 Mut 4 | SEQ ID NO:5 |
| Mut 2.2 | Region 2 Mut 2 | SEQ ID NO:8 |
| Mut 2.4 | Region 2 Mut 4 | SEQ ID NO:9 |
| Mut 3.9 | Region 3 Mut 9 | SEQ ID NO:12 |
| Mut 1.4-2.1 | Region 1 Mut 4 and Region 2 Mut 1 | SEQ ID NOs: 5 & 7 |
| Mut 1.5-2.2 | Region 1 Mut 5 and Region 2 Mut 2 | SEQ ID NOS:6 & 8 |
| Mut 1.5-2.4 | Region 1 Mut 5 and Region 2 Mut 4 | SEQ ID NOs:6 & 9 |
| Mut 2.1-3.9 | Region 2 Mut 1 and Region 3 Mut 9 | SEQ ID NOs:7 & 12 |
| Mut 2.2-3.9 | Region 2 Mut 2 and Region 3 Mut 9 | SEQ ID NOs:8 & 12 |
| Mut 2.4-3.9 | Region 2 Mut 4 and Region 3 Mut 9 | SEQ ID NOs:9 & 12 |

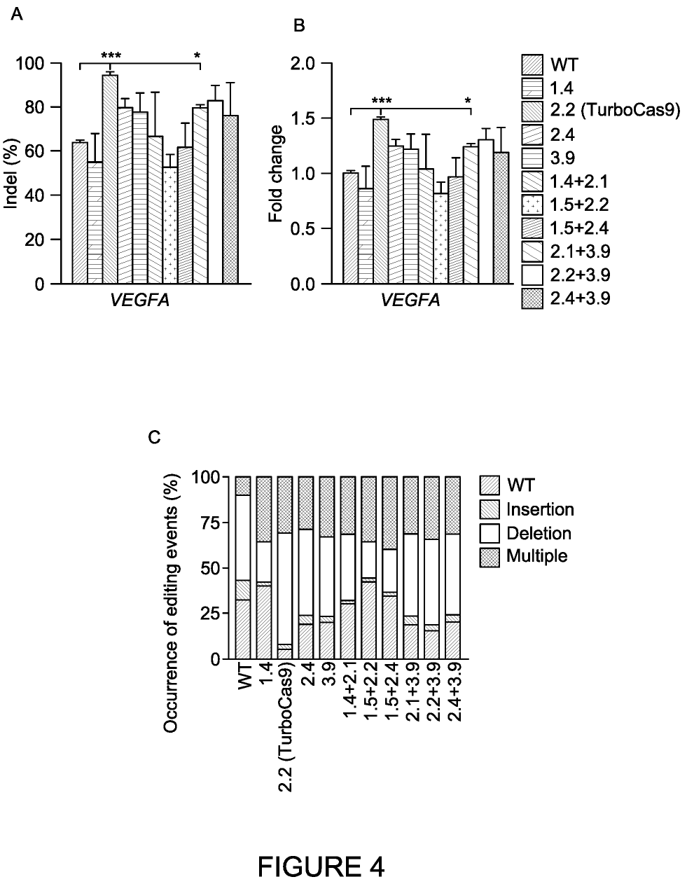

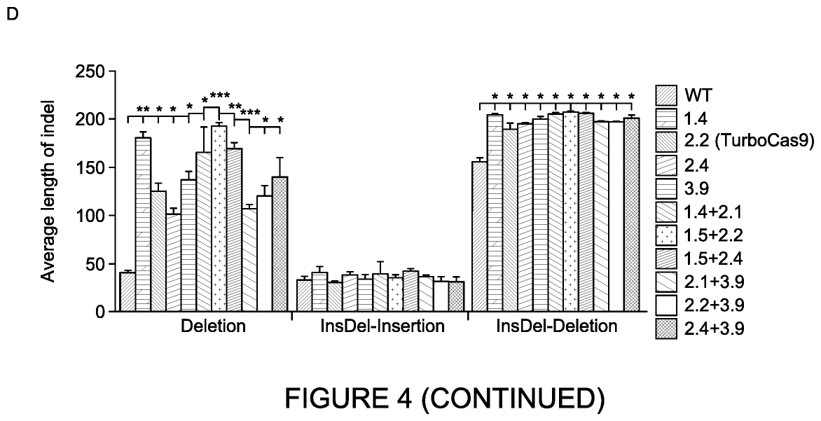

These mutants were codon optimized for mammalian-cell expression. The inventors used a well-characterized VEGFA gRNA, with known off target cleavage sites, and determined editing efficiencies in human HEK293T cells by next-generation sequencing of targeted DNA amplicons. Several mutants showed a significant decrease in the number of full-length reads corresponding to the wild-type VEGFA sequence, particularly mutants 2.2 and 2.1-3.9, with only 5% and 21%, respectively, of unedited VEGFA alleles remaining (FIG. 4A), whereas wild-type Cas9 failed to mutate 36% of VEGFA alleles. This result represents a 1.5-fold change in editing for mutant 2.2 and a 1.2-fold change for mutant 2.1-3.9 (FIG. 4B). Several other Cas9 mutants trended towards improved editing, but these were not statistically significant, while the others remained as active as the wild-type Cas9.

The inventors developed a computational pipeline to classify editing into three broad categories: single events of either a deletion or insertion, combined events in which an insertion and deletion or multiple thereof occurred within the same allele. Wild-type Cas9-mediated editing resulted predominantly in single deletion and insertion events; however, combined events were comparatively sparse (FIG. 4C). Single deletion events occurred at a similar rate for the designed Cas9 enzymes and were not significantly different to wild-type Cas9. The tested mutants had a roughly twofold decrease in the number of insertions (FIG. 4C), although the insertion lengths were similar (data not shown). Overall, the mutants caused a dramatic threefold or more increase in the number of multiply edited alleles (FIG. 4C). The accumulation of indels has been shown to be dependent on the cutting rate of editing enzymes (Brinkman et al., 2018, Mol Cell 70:801-813), indicating that the designed mutations successfully increased the activity of Cas9. Furthermore, in addition to the number of resulting mutations, every one of the engineered Cas9 enzymes induced significantly larger deletions (FIG. 4D). Increases in the sizes of the deletions for single events ranged from twofold for mutant 2.4 to well over fourfold for mutant 1.5-2.2.

Increasing Cas9 activity would result in a requirement for an increased number of repair events and thus potentially increase the complexity of DNA repair outcomes at these sites. To examine the nature of the induced mutations in more detail, the inventors mapped the exact locations and lengths of mutations and categorized indel events based on their respective CIGAR (concise idiosyncratic gapped alignment report) complexity level, where the higher the CIGAR complexity (CC) levels comprise deletions and insertions occurring simultaneously in more complex combinations. CC level 1 comprises all full length aligned wild-type sequences, CC2 are all soft clipped reads which were excluded from our analysis. CC3 are single insertion or deletion event and CC4 contains combined events with a single deletion and insertion. CC5 and above are of increasing complexity and comprise alleles with deletions and insertions occurring simultaneously, in varying numbers and in different combinations.

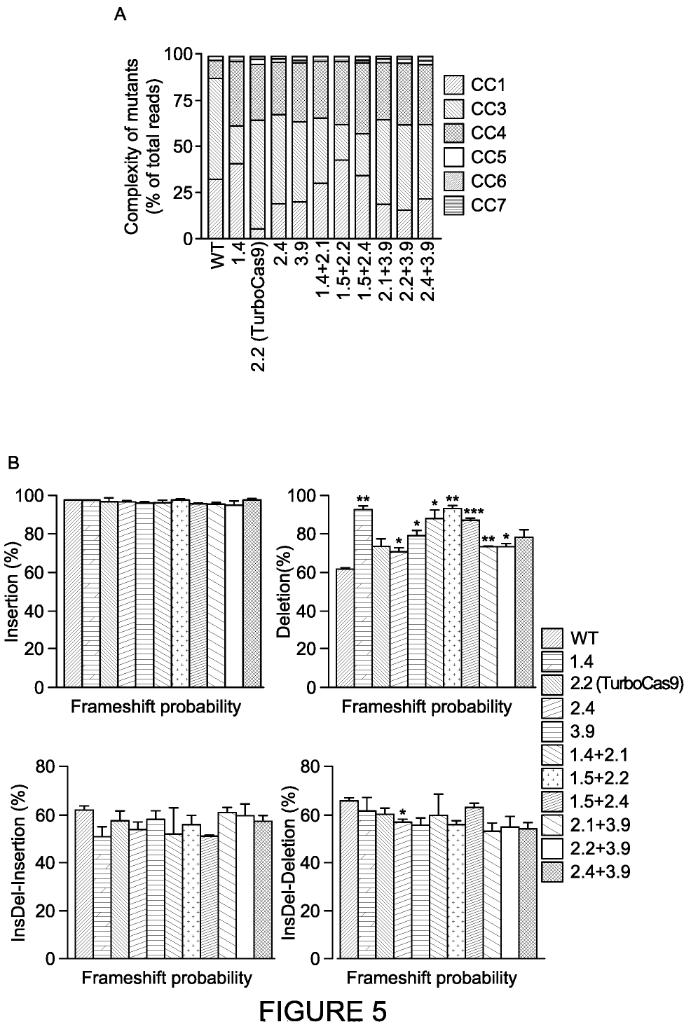

The inventors observed that the number of reads categorized in these higher CIGAR complexity levels in the tested mutants was significantly increased relative to the wild-type Cas9 (FIG. 5A). All mutants were found to have at least a twofold increase in the number of reads in CC4, mutant 2.2 had a roughly threefold increase in the number of reads present in CC6 and CC7, and mutants 2.2, 1.5-2.4 and 2.1-3.9 were found to have a significant increase of alleles in CC7 (FIG. 5A), which include multiple deletions and multiple insertions within a single allele. No significant change was found in the occurrence of frameshifts as a result of all editing events combined (data not shown), although the larger single deletion events induced by the engineered enzymes resulted in significantly more frameshifts in 8 out of 10 mutants (FIG. 5B). Several of the mutants trended towards or had significantly increased activity relative to wild-type Cas9, however, all mutants increased the number of complex editing events in comparison to wild-type Cas9. Structural studies have shown that Cas9 positions its gRNA and target DNA prior to reorientation of the HNH domain for cleavage (Jiang et al., 2015, Science 348:1477-1481). Displacement of the non-target strand and R-loop formation then enable cleavage by the HNH domain (Jiang et al., 2016, Science 351:867-871). Without wishing to be bound by theory, the inventors suggest that for mutants with the same number of cleaved alleles but more extensively mutated targets (such as Mut 1.4) the mutations may enhance cleavage without improving R-loop formation, while for others (such as Mut 2.2) both binding and cleavage appear to be enhanced. Taken together, the inventors conclude that the mutations significantly increase Cas9 activity as well as improving the enzyme's ability to generate indels that create a knockout or delete larger parts of target genes.

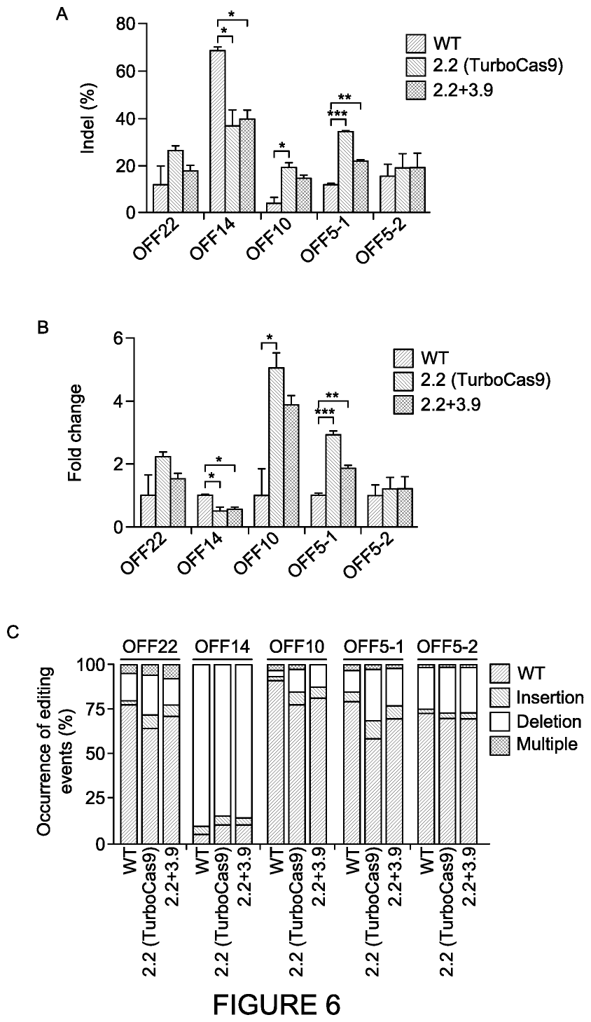

Increased fidelity has been observed to be reversely correlated with on-target activity (Liu et al., 2020, Nat Commun 11:6073). The inventors therefore examined whether mutants that have an increase in on-target activity would exhibit a similar increase in off-target activity. The top 5 known off-target sites for the VEGFA gRNA, named OFF22, OFF14, OFF10, OFFS-1 and OFFS-2, were amplified after editing by mutants 2.2 and 2.2-3.9, compared to wild-type Cas9. Interestingly, it was observed that the mutants increased editing at two off targets but did not significantly increase editing at two other off-targets, while one off-target had significantly fewer edits (FIGS. 6A and 6B). OFFS-2 differs from the VEGFA gRNA by two bp with one mismatch occurring at base 18 of the seed sequence, which is typically less tolerated by Cas9 and corroborated in the present data by the low levels of editing for the wild-type Cas9. The increased activity of mutants 2.2 and 2.2-3.9 does not seem to have lessened the fidelity of Cas9 when mismatches between the seed sequence and the target occur near the PAM sequence. OFF22 has a mismatch at bp 14 of the gRNA sequence and no significant difference was observed between tested mutants and wild-type Cas9. Interestingly, for OFF14 the tested mutants were found to have less activity than the wild-type Cas9. OFF10 and OFFS-1 were both found to have been edited significantly more by the mutants and both have mutations in the first 10 bp of the gRNA. Unlike the on-target site, the inventors did not observe an increase in multiply edited alleles nor a reduction in insertions for these off-target sites (FIG. 6C). Similar observations were found for the distribution of reads in the different levels of CIGAR complexity (FIG. 6C). Interestingly, the previously seen increase in deletion size for both the single deletions and also deletions within multiply edited alleles for the engineered Cas9 enzymes was not observed for off-targets. On the contrary, for several of the off-target sites a significant decrease in deletion size was observed. Thus, the tested mutants significantly increase Cas9 on-target activity without a consistent negative impact on fidelity.

### Example 5—Enhanced Nickase Activity in Mammalian Cells

Base editing genome editing technologies use the fusion of deaminase domains to CRISPR enzymes to enable the introduction of point mutations in DNA without generating double strand breaks. The technology typically uses the D10A mutation in the RuvC domain of Cas9 to generate a nickase; which then relies on cleavage by the HNH domain to generate a single stranded nick. Repair of the nicked strand then biases incorporation of deaminated DNA bases and thus the introduction of point mutations into the genome. Two major classes of base editors have been developed: cytidine base editors (CBEs), producing C to T transitions, and adenine base editors (ABEs), producing A to G transitions.

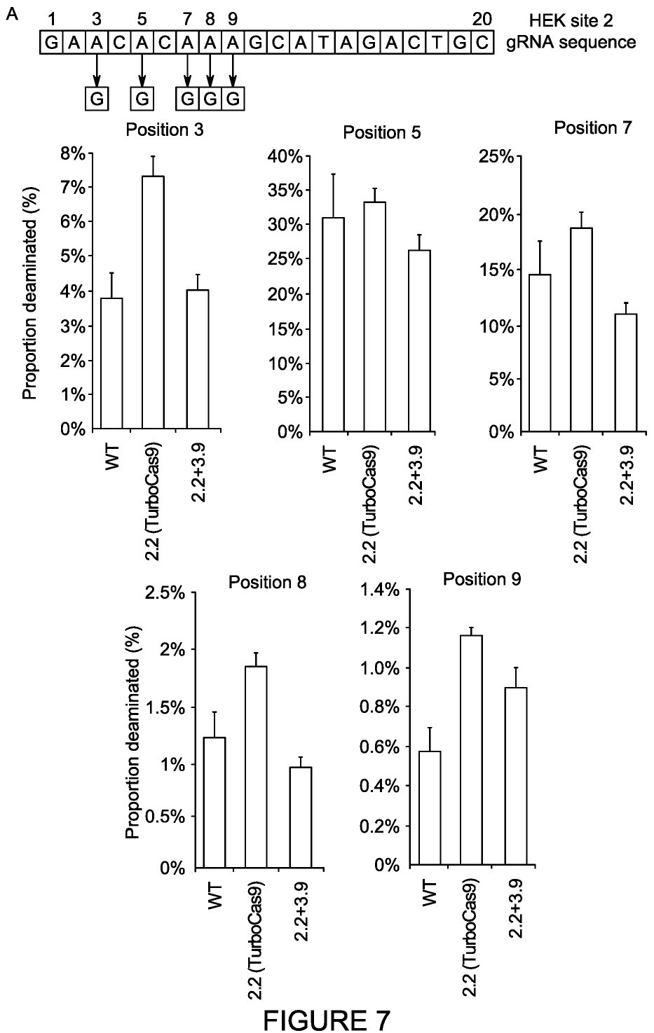

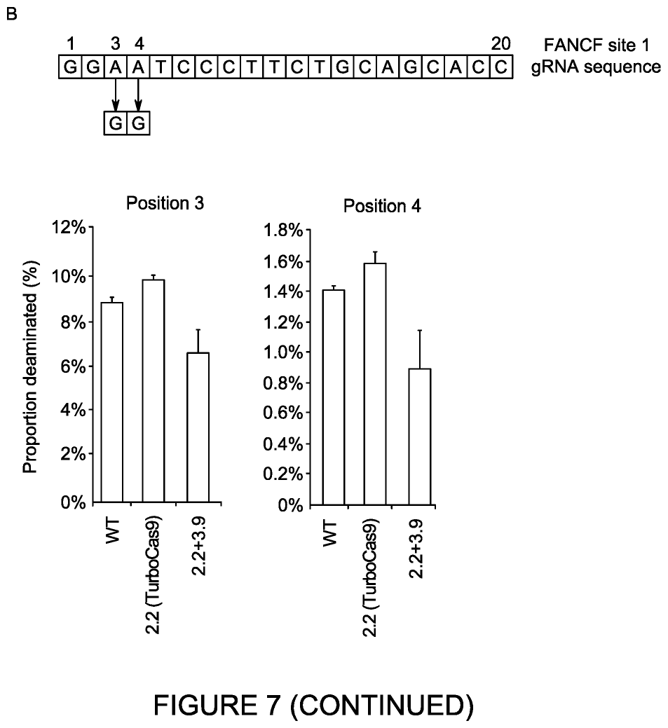

The inventors investigated the ability of mutants Mut 2.2 and 2.2-3.9 to enhance base editing, via increased nickase activity of the HNH domain, in the context of ABEs in HEK239T cells. Mut 2.2 (TurboCas9) enhanced base editing at sites targeted by both HEK site 2 and FANCF site 1 gRNAs (FIG. 7), demonstrating that enhanced nickase activity via activity enhancing Cas9 mutations can be valuable tools for genome editing.

1. An isolated Cas9 protein comprising SEQ ID NO:1 or a sequence at least 80% identical thereto, wherein:
the amino acid residues at positions 765 to 780 are replaced by the amino acid sequence of SEQ ID NO:5 or SEQ ID NO:6;
the amino acid residues at positions 838 to 853 are replaced by the amino acid sequence of SEQ ID NO:7, SEQ ID NO:8, SEQ ID NO:9 or SEQ ID NO:10; and/or
the amino acid residues at positions 911 to 925 are replaced by the amino acid sequence of SEQ ID NO:11, SEQ ID NO:12 or SEQ ID NO:13.

2. A Cas9 protein according to claim 1, wherein:
(i) the amino acid residues at positions 765 to 780 are replaced by the amino acid sequence of SEQ ID NO:5 or SEQ ID NO:6 and the amino acid residues at positions 838 to 853 are replaced by the amino acid sequence of SEQ ID NO:7, SEQ ID NO:8, SEQ ID NO:9 or SEQ ID NO:10;
(ii) the amino acid residues at positions 765 to 780 are replaced by the amino acid sequence of SEQ ID NO:5 and the amino acid residues at positions 911 to 925 are replaced by the amino acid sequence of SEQ ID NO:11 or SEQ ID NO:13; or
(iii) the amino acid residues at positions 765 to 780 are replaced by the amino acid sequence of SEQ ID NO:6 and the amino acid residues at positions 911 to 925 are replaced by the amino acid sequence of SEQ ID NO:11 or SEQ ID NO:12.

3-4. (canceled)

5. A Cas9 protein according to claim 1, wherein the amino acid residues at positions 838 to 853 are replaced by the amino acid sequence of SEQ ID NO:7, SEQ ID NO:8, SEQ ID NO:9 or SEQ ID NO:10 and the amino acid residues at positions 911 to 925 are replaced by the amino acid sequence of SEQ ID NO:11, SEQ ID NO:12 or SEQ ID NO:13.

6. A Cas9 protein according to claim 1, wherein:
the amino acid residues at positions 765 to 780 are replaced by the amino acid sequence of SEQ ID NO:5 or SEQ ID NO:6;
the amino acid residues at positions 838 to 853 are replaced by the amino acid sequence of SEQ ID NO:7, SEQ ID NO:8, SEQ ID NO:9 or SEQ ID NO:10; and
the amino acid residues at positions 911 to 925 are replaced by the amino acid sequence of SEQ ID NO:11, SEQ ID NO:12 or SEQ ID NO:13.

7. A Cas9 protein according to claim 6, wherein:
(i) the amino acid residues at positions 765 to 780 are replaced by the amino acid sequence of SEQ ID NO:5, the amino acid residues at positions 838 to 853 are replaced by the amino acid sequence of SEQ ID NO:7, and the amino acid residues at positions 911 to 925 are replaced by the amino acid sequence of SEQ ID NO:13;
(ii) the amino acid residues at positions 765 to 780 are replaced by the amino acid sequence of SEQ ID NO:6, the amino acid residues at positions 838 to 853 are replaced by the amino acid sequence of SEQ ID NO:7, and the amino acid residues at positions 911 to 925 are replaced by the amino acid sequence of SEQ ID NO:11;
(iii) the amino acid residues at positions 765 to 780 are replaced by the amino acid sequence of SEQ ID NO:6, the amino acid residues at positions 838 to 853 are replaced by the amino acid sequence of SEQ ID NO:8, and the amino acid residues at positions 911 to 925 are replaced by the amino acid sequence of SEQ ID NO:11; or
(iv) the amino acid residues at positions 765 to 780 are replaced by the amino acid sequence of SEQ ID NO:6, the amino acid residues at positions 838 to 853 are replaced by the amino acid sequence of SEQ ID NO:8, and the amino acid residues at positions 911 to 925 are replaced by the amino acid sequence of SEQ ID NO:12.

8-10. (canceled)

11. A Cas9 protein according to claim 1, wherein the amino acid residues at positions 838 to 853 are replaced by the amino acid sequence of SEQ ID NO:8.

12. An isolated Cas9 protein comprising an HNH domain comprising the amino acid sequence of SEQ ID NO:14 or a sequence at least 80% identical thereto, wherein:
the amino acid residues at positions 1 to 16 are replaced by the amino acid sequence of SEQ ID NO:5 or SEQ ID NO:6;
the amino acid residues at positions 74 to 89 are replaced by the amino acid sequence of SEQ ID NO:7, SEQ ID NO:8, SEQ ID NO:9 or SEQ ID NO:10; and/or
the amino acid residues at positions 147 to 161 are replaced by the amino acid sequence of SEQ ID NO:11, SEQ ID NO:12 or SEQ ID NO:13.

13. A Cas9 protein according to claim 12, wherein:
(i) the amino acid residues at positions 1 to 16 are replaced by the amino acid sequence of SEQ ID NO:5 or SEQ ID NO:6 and the amino acid residues at positions 74 to 89 are replaced by the amino acid sequence of SEQ ID NO:7, SEQ ID NO:8, SEQ ID NO:9 or SEQ ID NO:10;
(ii) the amino acid residues at positions 1 to 16 are replaced by the amino acid sequence of SEQ ID NO:5 and the amino acid residues at positions 147 to 161 are replaced by the amino acid sequence of SEQ ID NO:11 or SEQ ID NO:13; or
(iii) the amino acid residues at positions 1 to 16 are replaced by the amino acid sequence of SEQ ID NO:6 and the amino acid residues at positions 147 to 161 are replaced by the amino acid sequence of SEQ ID NO:11 or SEQ ID NO:12.

14-15. (canceled)

16. A Cas9 protein according to claim 12, wherein the amino acid residues at positions 74 to 89 are replaced by the amino acid sequence of SEQ ID NO:7, SEQ ID NO:8, SEQ ID NO:9 or SEQ ID NO:10 and the amino acid residues at positions 147 to 161 are replaced by the amino acid sequence of SEQ ID NO:11, SEQ ID NO:12 or SEQ ID NO:13.

17. A Cas9 protein according to claim 12, wherein:
the amino acid residues at positions 1 to 16 are replaced by the amino acid sequence of SEQ ID NO:5 or SEQ ID NO:6;
the amino acid residues at positions 74 to 89 are replaced by the amino acid sequence of SEQ ID NO:7, SEQ ID NO:8, SEQ ID NO:9 or SEQ ID NO:10; and
the amino acid residues at positions 147 to 161 are replaced by the amino acid sequence of SEQ ID NO:11, SEQ ID NO:12 or SEQ ID NO:13.

18. A Cas9 protein according to claim 17, wherein:
(i) the amino acid residues at positions 1 to 16 are replaced by the amino acid sequence of SEQ ID NO:5, the amino acid residues at positions 74 to 89 are replaced by the amino acid sequence of SEQ ID NO:7, and the amino acid residues at positions 147 to 161 are replaced by the amino acid sequence of SEQ ID NO:13;
(ii) the amino acid residues at positions 1 to 16 are replaced by the amino acid sequence of SEQ ID NO:6, the amino acid residues at positions 74 to 89 are replaced by the amino acid sequence of SEQ ID NO:7, and the amino acid residues at positions 147 to 161 are replaced by the amino acid sequence of SEQ ID NO:11,
(iii) the amino acid residues at positions 1 to 16 are replaced by the amino acid sequence of SEQ ID NO:6, the amino acid residues at positions 74 to 89 are replaced by the amino acid sequence of SEQ ID NO:8, and the amino acid residues at positions 147 to 161 are replaced by the amino acid sequence of SEQ ID NO:11, or
(iv) the amino acid residues at positions 1 to 16 are replaced by the amino acid sequence of SEQ ID NO:6, the amino acid residues at positions 74 to 89 are replaced by the amino acid sequence of SEQ ID NO:8, and the amino acid residues at positions 147 to 161 are replaced by the amino acid sequence of SEQ ID NO:12.

19-21. (canceled)

22. A Cas9 protein according to claim 12, wherein the amino acid residues at positions 74 to 89 are replaced by the amino acid sequence of SEQ ID NO:8.

23. A Cas9 protein according to claim 12, wherein the HNH domain is derived from the Cas9 protein of Streptococcus pyogenes.

24. A Cas9 protein according to claim 1, wherein the Cas9 protein is derived from the Cas9 protein of Streptococcus pyogenes.

25. An isolated polynucleotide encoding a Cas9 protein according to claim 1.

26. A vector comprising a polynucleotide according to claim 25.

27. A complex comprising: a Cas9 protein according to claim 1 and an associated guide RNA (gRNA).

28. A complex comprising: a Cas9 protein comprising an HNH domain according to claim 12 and an associated guide RNA (gRNA).

29. An isolated polynucleotide encoding a Cas9 protein comprising an HNH domain according to claim 12.

30. A vector comprising a polynucleotide according to claim 29.
